(19) United States
(12) Patent Application Publication
(10) Pub. No.: US 2006/0052390 A1
(43) Pub. Date: Mar. 9, 2006

(54) Treatment of multiple myeloma by p38 MAP kinase and proteasome inhibition

(75) Inventors: George F. Schreiner, Los Altos, CA (US); Andrew A. Protter, Palo Alto, CA (US); Linda S. Higgins, Palo Alto, CA (US)

Correspondence Address: MORRISON & FOERSTER LLP, 12531 HIGH BLUFF DRIVE, SUITE 100, SAN DIEGO, CA, 92130-2040, US

(73) Assignee: Scios, Inc., Fremont, CA (US)

(21) Appl. No.: 11/208,055

(22) Filed: Aug. 19, 2005

Related U.S. Application Data
(63) Continuation-in-part of application No. 11/024,169, filed on Dec. 27, 2004.
(60) Provisional application No. 60/532,440, filed on Dec. 24, 2003.
(60) Provisional application No. 60/633,979, filed on Dec. 6, 2004.

(51) Int. Cl. A61K 31/496; A61K 31/454

(52) U.S. Cl. 514/254.06; 514/322

(57) ABSTRACT

The present invention provides a method to treat multiple myeloma by the co-administration of one or more p38 MAP kinase inhibitors with one or more proteosome inhibitors.

### RELATED APPLICATIONS

This is a continuation-in-part of Ser. No. 11/024,169, filed Dec. 27, 2004, which claims the benefit of priority of U.S. Provisional Patent Application No. 60/532,440, filed Dec. 24, 2003, and U.S. Provisional Patent Application No. 60/633,979, filed Dec. 6, 2004, all of which are hereby incorporated by reference in their entirety.

### FIELD OF THE INVENTION

The present invention provides a method to treat multiple myeloma using p38 MAP kinase inhibitors and proteasome inhibitors.

### BACKGROUND OF THE INVENTION

There are approximately 45,000 people in the United States living with multiple myeloma and an estimated 14,600 new cases of multiple myeloma are diagnosed each year. New cases of multiple myeloma thus represent twenty percent of blood cancers and one percent of all types of cancer. There is an annual incidence of multiple myeloma of approximately 4 in 100,000 in the United States. The prognosis for individuals diagnosed with multiple myeloma varies. The median survival with conventional therapies like dexamethasone, melphalan, prednisone, and bisphosphates, is about 2.5 to 3 years. Individuals treated with high dose chemotherapy and bone marrow transplant show a 5-year survival rate of greater than 50%. Although the cause of multiple myeloma is not known, risk factors for developing multiple myeloma include exposure to atomic radiation, petroleum products, pesticides, solvents, heavy metals and airborne particles.

Multiple myeloma (MM) is a neoplastic disease involving malignant plasma cells. These malignant plasma cells accumulate in bone marrow and typically produce monoclonal IgG or IgA molecules. Individuals suffering from multiple myeloma often experience anemia, osteolytic lesions, renal failure, hypercalcemia, and recurrent bacterial infections. Individuals with multiple myeloma frequently present increased monoclonal plasma cells in their bone marrow and serum or urinary monoclonal protein. For a general review of MM, see Bataille & Harousseau, JAMA (1997) 336(23):1657-1664.

Typically, a small number of long-lived plasma cells in the bone marrow produce most of the IgG and IgA molecules found in blood serum. These plasma cells are well differentiated and do not divide. Phenotypically, plasma cells are CD38^{bright}, syndecan-1^{bright}, CD19⁺, and CD56^{weak/-}. Precursors to the plasma cells are plasmablasts that migrate from the lymph nodes after antigen stimulation to the bone marrow. Once arriving in a germinal center, these stimulated cells switch from immature IgM production to IgG or IgA. After the stimulated cells enter the bone marrow, they stop dividing and differentiate into plasma cells. Plasma cells usually undergo apoptosis and die after several weeks or months.

Myeloma cells, in contrast to the normal plasma cells, display a phenotype reminiscent of the immature plasmablast. The myeloma cells usually display CD38, syndecan-1, CDT9, and CD56^{bright}, and produce low amounts of immunoglobulins. Typically, the myeloma cells are aneuploid (hypoploid, but more often hyperploid) and their chromosomes have numerous structural abnormalities. Abnormalities are frequently apparent on chromosomes 13 (13q⁻) and 14 (14q⁺). The phenotypic characteristics of cellular immaturity and the 13q⁻ and 14q⁺ abnormalities correlate with resistance to treatment and to short survival characteristics of an aggressive disease state.

The myeloma cells adhere to and activate bone marrow stromal cells (BMSC) and are long-lived. Myeloma cells are dependent on interleukin-6 (IL-6), which is produced in copious quantities by BMSC. Interleukin-6 promotes MM cell growth. Hideshima, et al., Blood (2003) 101(2):703-705. Other cytokines are thought to be involved in the growth, survival, migration and adherence of MM cells and the development of osteolytic lesions. For example, vascular endothelial growth factor (VEGF) induces MM cell migration. Id. Adherence of MM cells to bone marrow stromal (BMSC) cells up-regulates IL-6 and VEGF secretion from both MM and BMSC cells. It is the interaction of the MM cells with the BMSC that is thought to trigger cytokine production and release from the BMSC.

The p38 mitogen-activated protein kinase (MAPK), which is a member of the MAPK family of kinases that is activated by cytokines and growth factors, may play a role in the multiple myeloma disease state. The exact role of p38 in MM, however, is unknown. Mitogen-activated protein kinase (MAPK) p38α mediates the cellular response to stress and is activated by pro-inflammatory cytokines such as TNFα and IL-1β. Activation of p38 in BMSC is required for the synthesis and secretion of IL-6, which is crucial for MM growth and promotes MM cell migration and drug resistance. In addition, p38 activation also induces cytokines which promote osteoclast differentiation and activation, likely contributors to the osteolytic lesions characteristic of MM. p38 inhibition suppresses the tumor-supportive state of BMSC in culture and has been proposed as a novel treatment for MM and for bone lesions associated with metastatic cancer. Since p38 inhibition targets the microenvironment, co-therapy with agents targeting MM tumor cells has been proposed as an attractive potential for effective treatment. One recent study demonstrated that a specific inhibitor of p38 MAPK inhibited IL-6 and vascular endothelial growth factor (VEGF) secretion in bone marrow stromal cells (BMSCs) without affecting the viability of these cells (Hideshima, et al., BLOOD (2003) 101(2):703-705). TNF-alpha-induced IL-6 secretion from BMSCs was also inhibited by the specific p38 MAPK inhibitor.

Proteasome inhibition has emerged as a promising novel anti-cancer therapy primarily for its preferential targeting of neoplastic cells relative to non-transformed cells. The ubiquitin-proteasome pathway regulates a number of important cellular processes critical for maintaining cell homeostasis and tissue physiology including cell cycle progression, apoptosis, inflammation, cell adhesion, migration, transcription and angiogenesis. Proteasome inhibitors trigger apoptosis in a variety of tumor-derived cell lines and patient-derived cells, many of which are hematopoietic in origin, including monoblasts, T-cell and lymphocytic leukemia, promyelocytic leukemia, lymphoma and multiple myeloma cells. Proteasome inhibitors induce the accumulation of p21 or p27 in these cells, causing arrest in the cell cycle and subsequent apoptosis, even in cells with mutated or transcriptionally inactive p53. Proteasome inhibitors also induce the accumulation of Bax, overcome Bcl-2 mediated protection and enhance the intrinsic apoptotic pathway mediated by cytochrome c.

Certain proteasome inhibitors have recently been shown to have positive effects on MM cells and on the microenvironment through overlapping but non-identical mechanisms to p38 inhibition. For example, a recently published study reported that the proteasome inhibitor PS-431 (BORTEZOMIB/VELCADE) potentiates sensitivity of multiple myeloma cells to conventional chemotherapeutic agents. Mitsiades, et al., BLOOD (2003) 101:2377-2380. Additionally, the Food and Drug Administration announced on May 13, 2003, that the proteasome inhibitor VELCADE (bortezomib) was approved to treat patients with MM whose disease has relapsed after two prior treatments and who have demonstrated resistance to their last treatment for late stage MM patients. It represents a new treatment paradigm that targets not only the tumor cell directly, but also the supporting bone marrow (BM) milieu and MM cell-host interaction. PS-341 promotes MM apoptosis by activating caspases-8, -9 and -3, through a process that requires JNK, and impairs DNA repair by the cleavage of DNA protein kinase catalytic subunit (DNA-PKcs) and ATM.

PS-341 also inhibits the transcription and secretion of IL-6 in the bone marrow milieu and abrogates the adherence of MM to BMSCs. PS-341 additionally inhibits IL-6-triggered signaling pathways that promote MM cell growth and proliferation, via a caspase-mediated down-regulation of gp130, a downstream target of the IL-6 receptor, gp80. These actions presumably underlie responses of patients, a small proportion of which are complete responses. While impressive responses can be achieved with VELCADE in some patients, many do not respond and side effect incidence is high.

Recent reports have shown that heat shock protein 27 (Hsp27), a downstream target of p38, has a role in conferring dexamethasone-resistance to MM cells. In addition, overexpression of Hsp27 confers PS-341 resistance to some lymphoma cells. Hsp27 belongs to a family of proteins that are induced upon cytotoxic insult, providing a cytoprotective effect that enhances the cell's ability to survive. Hsp27 acts as a molecular chaperone that can directly interfere with the mechanisms of caspase activation, thus playing a critical role in the regulation of the cell's apoptotic machinery. Hsp27 function is also modulated by its phosphorylation state. During stress, the increase in Hsp27 levels is normally preceded by a phosphorylation-induced reorganization of the protein's multimeric status. Higher levels of Hsp27 are commonly detected in a variety of cancers including breast, prostate, ovarian, as well as Hodgkin's disease, and this renders tumors more drug-resistant and confers increased metastatic potential. Thus, the use of various approaches to eliminate the expression of Hsp27 is being explored for the treatment of Hsp27-expressing cancers.

### SUMMARY OF THE INVENTION

The invention is directed to methods useful in treating multiple myeloma (MM) by combining one or more p38 MAPK inhibitors, with one or more proteasome inhibitors. A role for p38 kinase inhibition as a treatment modality for combating multiple myeloma is discussed herein. In a preferred embodiment, small molecule antagonists of p38 MAP kinase are used to treat multiple myeloma, preferably in combination with a proteasome inhibitor.

### BRIEF DESCRIPTION OF THE DRAWINGS

FIG. 1 shows a graphic representation of the effect of a p38 MAPK inhibitor on p38 MAPK phosphorylation in MM cells.

FIG. 2 shows a bar graph showing the effect of a p38 MAPK inhibitor on basal p38 phosphorylation levels in MM cell lines.

FIG. 3 shows the effect of a p38 MAPK inhibitor on p38α MAPK activation and activity in BMSC cells.

FIG. 4 shows the lack of effect of a p38 MAPK inhibitor on BMSC viability.

FIG. 5 shows bar graphs illustrating the reduction of RANKL mRNA induction in BMSC and BMSC/MM cultures as a result of treatment with a p38 MAPK inhibitor.

FIG. 6A-D show that Compound 57 enhances the anti-proliferative effect of proteasome inhibitor MG-132 on MM cells.

FIG. 7A-F show that Compound 57 enhances the anti-proliferative effect of proteasome inhibitor MG-132 on MM cells co-cultured with BMSC cells.

### DETAILED DESCRIPTION OF THE PREFERRED EMBODIMENTS

The invention described herein relates to the use of MAP kinase inhibitors, preferably p38 MAP kinase inhibitors, in combination with proteasome inhibitors to treat multiple myeloma (MM). Administration of MAP kinase inhibitors generally and p38 MAP kinase inhibitors particularly and proteasome inhibitors, deter MM cell proliferation. Inhibition of p38 MAP kinase activity in combination with the inhibition of proteasome activity has a number of direct and indirect effects on the MM cells that have therapeutic benefits for patients suffering from MM.

MAP Kinase Inhibitors, Cytokines and MM

Mitogen-activated protein kinases (MAPKs) are activated by tyrosine and threonine phosphorylation. The p38 MAPK protein kinase family is activated primarily by cellular stresses and not mitogenic stimuli. The activation domain of p38 contains the sequence TGY, which represent the tyrosine and threonine residues required for activation (targeted by MKK3 and MKK6). The physiological role of the different p38 isoforms (which are derived from three genes as well as differential splicing) is still unclear. Among the identified targets for p38 are MAPKAPK-2 and the transcription factors, CHOP/GADD153 (Wang and Ron, Science (1997) 272, 1347-1349), MEF2C (Han et al., Nature (1997), 386, 296-299) and ATF2.

The activation of p38 MAP kinase (phosphorylated form) in MM cells and in bone marrow stromal cells (BMSC) is induced by cytokines and other inflammatory moieties present in the MM bone marrow milieu. Activation of p38 MAP kinase may be induced even in unstimulated MM cells by tumor necrosis factor (TNF). This activation may result in the secretion of cytokines thought to be involved in the pathogenesis of MM. Certain cytokines play a role in promoting a bone marrow microenvironment that is hospitable to the growth, survival, and migration of MM cells. Further, the activated marrow microenvironment in MM supports the unusual drug resistance observed in MM compared to other B cell cancers such as leukemias. Interleukin-6 (IL-6) is thought to be a primary mediator in these effects and is produced by both MM and marrow cells. Additional cytokines thought to play roles in the pathology of MM microenvironment include interleukin-1 (IL-1), interleukin-11 (IL-11), tumor necrosis factor (TNF), insulin-like growth factor-1 (IGF-1), macrophage inflammatory protein-1 (MIP-1), receptor activator of NF-kappa B ligand (RANKL), and transforming growth factor-beta (TGF-β).

Administering MAP kinase inhibitors negatively impacts the bone marrow milieu in which MM cells propagate by altering cytokine expression. For example, p38 inhibitors act to reduce interleukin-6 (IL-6) production from bone marrow stromal cells (BMSCs). Production of IL-6 is thought to be important for maintaining a microenvironment that is favorable for multiple myeloma cell proliferation, that is, MM cell growth and replication. While the impact of p38 inhibitors on cytokine expression, such as IL-6 expression, is a likely mechanism by which to explain the therapeutic impact of p38 inhibitors on MM, it is not the only mechanism available to explain these positive effects. Accordingly, this mechanism is provided solely as a tool for conceptualizing the role that p38 inhibitors can play in treating MM and is not intended to be limiting in any way.

MAP Kinase Inhibitors and MM Cell Drug Resistance

Multiple myeloma is a difficult cancer to treat, in part because MM cells frequently develop resistance to standard chemotherapeutic agents. This is an extremely important point, since patients eventually become resistant to conventional therapy and their disease progresses. The marrow microenvironment in MM is thought to be responsible for the unusual resistance to chemotherapy observed for MM cells compared to other B cell cancers.

Multiple myeloma cells cultured from patients retain their initial or treatment induced resistance profile in many cases. IL-6 expression may be responsible, in part, for dexamethasone resistance of some MM cells. It has already been pointed out that inhibition of the MAP kinase p38 blocks MM cell and BMSC secretion of IL-6. p38 MAP kinase inhibition of IL-6 secretion may thus be an affective means by which to treat MM.

p38 activation may help MM cells adapt in a way that enhances MM cell survival. For example, activated heat shock protein 27 is thought to play a role in block apoptosis in MM cells. Activated p38 phosphorylates and activates HSP-27. By blocking the activation of HSP-27 through p38 inhibition, the postulated anti-apoptotic effect of HSP-27 may be removed. Thus, p38 inhibition may serve to render MM cells sensitive to chemotherapeutic agents, such as apoptosis-promoting agents, to which MM cells might otherwise be resistant.

MAP Kinase Inhibitors, Proteasome Inhibitors, and MM

The constant breakdown and resynthesis of bone is mediated by bone cells that are regulated by a large number of cytokines and growth factors. Cytokine production by BMSC's and MM cells may play an important role in maintaining a microenvironment within the bone milieu that promotes MM cell propagation. Proteasome inhibitors have been reported to block production of cytokines by MM cells and stromal cells.

The proteasome is a multisubunit complex responsible for the degradation of almost all cytosolic proteins. A number of proteasome inhibitors such as lactacystin have been identified. C-terminal peptide aldehydes were the first of several classes of compounds to be investigated as proteasome inhibitors. These inhibitors are cell-permeable and block proteasome function without affecting normal biological processes such as ATP metabolism and protein synthesis. M. Bogyo, M. Gaczynska and H. L. Ploegh, Biopoly., 43, 269-280 (1997). Specific examples of a variety of proteasome inhibitors include epoxomicin ((2R)-2-[Acetyl-(N-Methyl-L-Isoleucyl)-L-Isoleucyl-L-Threonyl-L-Leucyl]-2-Methyloxirane); Lactacystin (N-Acetyl-L-Cysteine, S-[2R,3 S,4R]-3-Hydroxy-2-[(1S)-1-Hydroxy-2-Methylpropyl]-4-Methyl-5-Oxo-2-Pyrolidinecarbonyl]); Z-Ile-Glu(OtBu)-Ala-Leu-H(Carbobenzoxy-L-Isoleucyl-Gamma-t-Butyl-L-Glutamyl-L-Alanyl-L-Leucinal; Z-Leu-Leu-Leu-H [MG 132](Carbobenzoxy-L-Leucyl-L-Leucyl-L-Leucinal); and Z-Leu-Leu-Nva-H [MG 115] (Carbobenzoxy-L-Leucyl-L-Leucyl-L-Norvalinal). Additional examples of proteasome inhibitors can be found in U.S. Pat. No. 6,645,999, entitled “Lactacystin Analogs,” which is hereby incorporated by reference.

Another effective proteasome inhibitor is thaliamide and its derivatives, such as REVIMID. Examples of these compounds are disclosed in U.S. Pat. No. 5,635,517, which is hereby incorporated by reference in its entirety.

Proteasome inhibition as a means of blocking cytokine production is thought to be mediated, in part, by blocking NFκB activation. (See U.S. patent application No. 20020111292, filed Jul. 15, 2002, published Aug. 15, 2002). p38 and NFκB both act at key control points in signaling cascades that result in integrated inflammatory responses characterized by cytokine and COX-2 production. p38 and NFκB can be activated independently or in a coordinated fashion. In some systems p38 activation leads to NFκK activation, and cross-talk between the two systems may be common. Patents relevant to VELCADE and its use include U.S. Pat. Nos. 5,780,454, 6,083,903, 6,297,217, 6,617,317 6,713,446, and 6,747,150, each of which is incorporated by reference in their entirety.

VELCADE (bortezombid) was recently approved by the United Stated Food and Drug Administration (FDA) for treatment of relapsed refractory MM. Based on preclinical data, VELCADE (bortezombid) is thought to reduce cytokine production in the microenvironment and by directly killing myeloma cells. VELCADE (bortezombid) is effective in only about a third of patients, however, and of this third complete responses are very rare. In addition, a fully effective pharmacological dose (based on proteasome inhibition assayed in blood) is not achievable due to dose limiting toxicities and associated side effects such as neuropathy.

VELCADE (bortezombid) inhibits the proteasome, with one consequence being reduced degradation of the NFκK inhibitory protein IκK, which results in a reduction of NFκK activity. Activation of these pathways result in secretion of cytokines involved in MM pathology when activated in BMSC and MM cells. Accordingly, inhibition of these two pathways should be effective in inhibiting MM cell propagation.

Perhaps more important, p38 MAP kinase inhibitors and proteasome inhibitors may act synergistically by making MM cells less resistant to proteasome inhibition. This proteasome inhibition may allow either a reduction in proteasome inhibitor dose while maintaining a given level of efficacy (such as when dose limited by a toxicity) or an increase in efficacy at a given dose. Drug interaction studies on MM cells cultured alone or in combination with BMSC show that exposure to p38 inhibition increases sensitivity to proteasome inhibition.

Because both p38 MAP kinase and particular proteasome inhibitors affect related systems, the combination of p38 inhibition and proteasome inhibition could be more effective than either agent through synergies of their overlapping mechanisms. For example, administering MAP kinase inhibitors, such as p38 MAP kinase inhibitors may sensitize MM cells to VELCADE, which would allow for lower doses of the proteasome inhibitor to be administered.

Neuropathic pain is a major dose limiting toxicity of VELCADE, as it is for many chemotherapeutic agents (see VELCADE package insert). Two related components are involved: actual neural injury, which may be irreversible, and the neuropathic pain. Protection from neural injury has been shown in a variety of preclinical models including nerve crush, brain ischemia, and excitatory amino acid neurotoxicity. There is literature surrounding these findings, including work in a stroke model (Koistinaho, et al., PNAS (2002)). Enhanced regrowth of severed peripheral nerves in the presence of a p38 inhibitor was shown to be relevant to protection as well as to recovery following neuropathy. Accordingly, as disclosed in U.S. patent application Ser. No. 10/655,745, which is hereby incorporated by reference in its entirety, the clear benefits of p38 MAP kinase inhibitor administration in several models of neuropathic pain has been shown.

MM Osteolytic Lesions, MAP Kinase Inhibitors, and Proteasome Inhibitors

Multiple myeloma cells upregulate osteolytic activity as they propagate in the bone milieu, forming osteolytic lesions. Bone erosion or osteolytic lesions typically begin intramedullarly and progresses through the cortex of the bone. Radiological analysis of multiple myeloma sites is characterized by the presence of irregular osteolytic lesions of different sizes in the involved bone. The destruction of calcified bone tissue can result in hypercalcemia, which may cause confusion, weakness, lethargy, spinal cord compression and renal insufficiency in a person suffering from MM.

Providing p38 MAP kinase inhibitors in combination with one or more proteasome inhibitors may provide an effective method of preventing or reducing MM-associated osteolytic activity. For example, in one theoretical model, administering p38 kinase inhibitors prevents or reduces the osteolytic activity of MM cells by altering nuclear factor-κB activity, which facilitates osteolytic activity.

Inhibitors of p38 MAP Kinase

As used herein, the term “inhibitor” includes, but is not limited to, any suitable molecule, compound, protein or fragment thereof, nucleic acid, formulation or substance that can regulate p38 MAP kinase activity. The data discussed herein can be reproduced using any disclosed p38 MAPK inhibitor. The inhibitor can affect a single p38 MAP kinase isoform (e.g., p38α, p38β, p38γ or p38δ), more than one isoform, or all isoforms of p38 MAP kinase. In a preferred embodiment, the inhibitor regulates the a isoform of p38 MAP kinase.

In a preferred embodiment of the disclosed invention, it is contemplated that the particular inhibitor can exhibit its regulatory effect upstream or downstream of p38 MAP kinase or on p38 MAP kinase directly. Examples of inhibitor regulated p38 MAP kinase activity include those where the inhibitor can decrease transcription and/or translation of p38 MAP kinase, can decrease or inhibit post-translational modification and/or cellular trafficking of p38 MAP kinase, or can shorten the half-life of p38 MAP kinase. The inhibitor can also reversibly or irreversibly bind p38 MAP kinase, inactivate its enzymatic activity, or otherwise interfere with its interaction with downstream substrates.

If acting on p38 MAP kinase directly, in one embodiment the inhibitor should exhibit an IC₅₀ value of about 5 μM or less, preferably about 500 nM or less, more preferably about 100 nM or less. In a related embodiment, the inhibitor should exhibit an IC₅₀ value relative to the p38α MAP kinase isoform that is about ten fold less than that observed when the same inhibitor is tested against other p38 MAP kinase isoforms in a comparable assay.

Those skilled in the art can determine whether or not a compound is useful in the disclosed invention by evaluating its p38 MAP kinase activity relative to its IC₅₀ value for p38 kinase. This evaluation can be accomplished through conventional in vitro assays. In vitro assays include assays that assess inhibition of kinase or ATPase activity of activated p38 MAP kinase. In vitro assays can also assess the ability of the inhibitor to bind to a p38 MAP kinase or to reduce or block an identified downstream effect of the activated p38 MAP kinase, e.g., cytokine secretion. IC₅₀ values are calculated using the concentration of inhibitor that causes a 50% decrease as compared to a control.

A binding assay is a fairly inexpensive and simple in vitro assay to run. As previously mentioned, binding of a molecule to p38 MAP kinase, in and of itself, can be inhibitory, due to steric, allosteric or charge-charge interactions. A binding assay can be performed in solution or on a solid phase using p38 MAP kinase or a fragment thereof as a target. By using this as an initial screen, one can evaluate libraries of compounds for potential p38 MAP kinase regulatory activity.

The target in a binding assay can be either free in solution, fixed to a support, or expressed in or on the surface of a cell. A label (e.g., radioactive, fluorescent, quenching, etc.) can be placed on the target, compound, or both to determine the presence or absence of binding. This approach can also be used to conduct a competitive binding assay to assess the inhibition of binding of a target to a natural or artificial substrate or binding partner. In any case, one can measure, either directly or indirectly, the amount of free label versus bound label to determine binding. There are many known variations and adaptations of this approach to minimize interference with binding activity and optimize signal.

For purposes of in vitro cellular assays, the compounds that represent potential inhibitors of p38 MAP kinase function can be administered to a cell in any number of ways. Preferably, the compound or composition can be added to the medium in which the cell is growing, such as tissue culture medium for cells grown in culture. The compound is provided in standard serial dilutions or in an amount determined by analogy to known modulators. Alternatively, the potential inhibitor can be encoded by a nucleic acid that is introduced into the cell wherein the cell produces the potential inhibitor itself.

Alternative assays involving in vitro analysis of potential inhibitors include those where cells (e.g., HeLa) transfected with DNA coding for relevant kinases can be activated with substances such as sorbitol, IL-1, TNF, or PMA. After immunoprecipitation of cell lysates, equal aliquots of immune complexes of the kinases are pre-incubated for an adequate time with a specific concentration of the potential inhibitor followed by addition of kinase substrate buffer mix containing labeled ATP and GST-ATF2 or MBP. After incubation, kinase reactions are terminated by the addition of SDS loading buffer. Phosphorylated substrate is resolved through SDS-PAGE and visualized and quantitated in a phosphorimager. The p38 MAP kinase regulation, in terms of phosphorylation and IC₅₀ values, can be determined by quantitation. See e.g., Kumar, S. et al., Biochem. Biophys. Res. Commun. 235:533-538 (1997). Similar techniques can be used to evaluate the effects of potential inhibitors on other MAP kinases.

Other in vitro assays can assess the production of TNF-α as a correlation to p38 MAP kinase activity. One such example is a Human Whole Blood Assay. In this assay, venous blood is collected from, e.g., healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific Co., San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 R of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath, and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.). IC₅₀ values are calculated using the concentration of inhibitor that causes a 50% decrease as compared to a control.

A similar assay is an Enriched Mononuclear Cell Assay. The enriched mononuclear cell assay begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well. After the cells have settled, the media is aspirated and new media containing 100 ng/ml of the cytokine stimulatory factor Lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an Enzyme Linked Immunoassay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

While IC₅₀ values are an initial indicia for identifying compounds that are useful for the invention, it is contemplated that one skilled in the art would further consider additional and conventional pharmaceutical considerations including but not limited to bioavailability, pK values, routes of delivery, solubility, and the like.

Exemplary Inhibitors

Preferred examples of the compounds of the invention are of the formula:

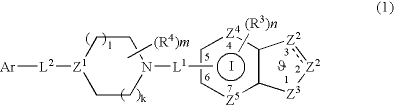

and the pharmaceutically acceptable salts thereof, or a pharmaceutical composition thereof, wherein 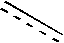
represents a single or double bond;

one Z² is CA or CR⁸A and the other is CR¹, CR¹₂, NR⁶ or N wherein each R¹, R⁶ and R⁸ is independently hydrogen or noninterfering substituent;

A is —Wᵢ—COXⱼY wherein Y is COR² or an isostere thereof and R² is hydrogen or a noninterfering substituent, each of W and X is a spacer of 2-6 Å, and each of i and j is independently 0 or 1;

Z³ is NR⁷ or O;

each of Z⁴ and Z⁵ is independently N or CR¹ wherein R¹ is as defined above and wherein at least one of Z⁴ and Z⁵ is N;

each R³ is independently a noninterfering substituent;

n is 0-3;

each of L¹ and L² is a linker;

each R⁴ is independently a noninterfering substituent;

m is 0-4;

Z¹ is CR⁵ or N wherein R⁵ is hydrogen or a noninterfering substituent;

each of 1 and k is an integer from 0-2 wherein the sum of 1 and k is 0-3;

Ar is an aryl group substituted with 0-5 noninterfering substituents, wherein two noninterfering substituents can form a fused ring.

Preferred embodiments of compounds useful in the invention are derivatives of indole-type compounds containing a mandatory substituent, A, at a position corresponding to the 2- or 3-position of indole. In general, an indole-type nucleus is preferred, although alternatives within the scope of the invention are also illustrated below. Additionally, PCT publication WO00/71535, published 7 Dec. 2000, discloses indole derived compounds that are specific inhibitors of p38 kinase. The disclosure of this document is incorporated herein by reference.

U.S. Provisional Patent Application No.60/417,599 filed 9 Oct. 2002 and U.S. patent application Ser. No. 10/683,656, filed Oct. 9, 2003, disclose azaindole derivatives that are useful in treating conditions that are characterized by enhanced p38 activity and are therefore useful for purposes of this invention.

As used herein, a “noninterfering substituent” is a substituent which either leaves the ability of the compound of formula (1) to inhibit p38-α activity qualitatively intact or enhances the activity of the inhibitor. Thus, the substituent may alter the degree of inhibition of p38 However, as long as the compound of formula (1) retains the ability to inhibit p38 activity, the substituent will be classified as “noninterfering.” As mentioned above, a number of assays for determining the ability of any compound to inhibit p38 activity are available in the art. A whole blood assay for this evaluation is illustrated below: the gene for p38 has been cloned and the protein can be prepared recombinantly and its activity assessed, including an assessment of the ability of an arbitrarily chosen compound to interfere with this activity. The essential features of the molecule are tightly defined. The positions which are occupied by “noninterfering substituents” can be substituted by conventional organic moieties as is understood in the art. It is irrelevant to the present invention to test the outer limits of such substitutions.

Regarding the compounds of formula (1), L¹ and L² are described herein as linkers. Typical linkers include alkylene, i.e. (CH₂)ₙ—R; alkenylene—i.e., an alkylene moiety which contains a double bond, including a double bond at one terminus. Other suitable linkers include, for example, substituted alkylenes or alkenylenes, carbonyl moieties, and the like.

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when so stated however, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, “inorganic residue” refers to a residue that does not contain carbon. Examples include, but are not limited to, halo, hydroxy, NO₂ or NH₂.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

When the compounds of Formula 1 contain one or more chiral centers, the invention includes optically pure forms as well as mixtures of stereoisomers or enantiomers.

With respect to the portion of the compound of formula (1) between the atom of Ar bound to L² and ring α, L¹ and L² are linkers which space the substituent Ar from ring α at a distance of 4.5-24 Å, preferably 6-20 Å, more preferably 7.5-10 Å. In a preferred embodiment, the distance of substituent Ar from ring is less than 24 Å. The distance is measured from the center of the α ring to the atom of Ar to which the linker L² is attached. Typical, but nonlimiting, embodiments of L¹ and L² are CO and isosteres thereof, or optionally substituted isosteres, or longer chain forms. L², in particular, may be alkylene or alkenylene optionally substituted with noninterfering substituents or L¹ or L² may be or may include a heteroatom such as N, S or O. Such substituents include, but are limited to, a moiety selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR², SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety.

Isosteres of CO and CH₂, include SO, SO₂, or CHOH. CO and CH₂ are preferred.

Thus, L² is substituted with 0-2 substituents. Where appropriate, two optional substituents on L² can be joined to form a non-aromatic saturated or unsaturated hydrocarbyl ring that includes 0-3 heteroatoms such as O, S and/or N and which contains 3 to 8 members. Two optional substituents on L2 can be joined to form a carbonyl moiety which can be subsequently converted to an oxime, an oximeether, an oximeester, or a ketal.

Ar is aryl, heteroaryl, including 6-5 fused heteroaryl, cycloaliphatic or cycloheteroaliphatic that can be optionally substituted. Ar is preferably optionally substituted phenyl.

Each substituent on Ar is independently a hydrocarbyl residue (1-20C) containing 0-5 heteroatoms selected from O, S and N, or is an inorganic residue. Preferred substituents include those selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two of said optional substituents on adjacent positions can be joined to form a fused, optionally substituted aromatic or nonaromatic, saturated or unsaturated ring which contains 3-8 members. More preferred substituents include halo, alkyl (1-4C) and more preferably, fluoro, chloro and methyl. These substituents may occupy all available positions of the aryl ring of Ar, preferably 1-2 positions, most preferably one position. These substituents may be optionally substituted with substituents similar to those listed. Of course some substituents, such as halo, are not further substituted, as known to one skilled in the art.

Two substituents on Ar can be joined to form a fused, optionally substituted aromatic or nonaromatic, saturated or unsaturated ring which contains 3-8 members.

Regarding formula (1), between L¹ and L² is a piperidine-type moiety of the following formula:

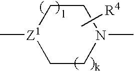

Z¹ is CR⁵ or N wherein R⁵ is H or a noninterfering substituent. Each of l and k is an integer from 0-2 wherein the sum of l and k is 0-3. The noninterfering substituents R⁵ include, without limitation, halo, alkyl, alkoxy, aryl, arylalkyl, aryloxy, heteroaryl, acyl, carboxy, or hydroxy. Preferably, R⁵ is H, alkyl, OR, NR₂, SR or halo, where R is H or alkyl. Additionally, R⁵ can be joined with an R⁴ substituent to form an optionally substituted non-aromatic saturated or unsaturated hydrocarbyl ring which contains 3-8 members and 0-3 heteroatoms such as O, N and/or S. Preferred embodiments include compounds wherein Z¹ is CH or N, and those wherein both l and k are 1.

R⁴ represents a noninterfering substituent such as a hydrocarbyl residue (1-20C) containing 0-5 heteroatoms selected from O, S and N. Preferably R⁴ is alkyl, alkoxy, aryl, arylalkyl, aryloxy, heteroalkyl, heteroaryl, heteroarylalkyl, RCO, ═O, acyl, halo, CN, OR, NRCOR, NR, wherein R is H, alkyl (preferably 1-4C), aryl, or hetero forms thereof. Each appropriate substituent is itself unsubstituted or substituted with 1-3 substituents. The substituents are preferably independently selected from a group that includes alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof. R⁴ may occur m times on the ring; m is an integer of 0-4. Preferred embodiments of R⁴ comprise alkyl (1-4C) especially two alkyl substituents and carbonyl. Most preferably R⁴ comprises two methyl groups at positions 2 and 5 or 3 and 6 of a piperidinyl or piperazinyl ring or ═O preferably at the 5-position of the ring. The substituted forms may be chiral and an isolated enantiomer may be preferred.

R³ also represents a noninterfering substituent. Such substituents include hydrocarbyl residues (1-6C) containing 0-2 heteroatoms selected from O, S and/or N and inorganic residues. n is an integer of 0-3, preferably 0 or 1. Preferably, the substituents represented by R³ are independently halo, alkyl, heteroalkyl, OCOR, OR, NRCOR, SR, or NR₂, wherein R is H, alkyl, aryl, or heteroforms thereof. More preferably R³ substituents are selected from alkyl, alkoxy or halo, and most preferably methoxy, methyl, and chloro. Most preferably, n is 0 and the α ring is unsubstituted, except for L¹ or n is 1 and R³ is halo or methoxy.

In the ring labeled [symbol] , Z³ may be NR⁷ or O—i.e., the compounds may be related to indole or benzofuran. If C³ is NR⁷, preferred embodiments of R⁷ include H or optionally substituted alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof. More preferably, R⁷ is hydrogen or is alkyl (1-4C), preferably methyl or is acyl (1-4C), or is COOR wherein R is H, alkyl, alkenyl of aryl or hetero forms thereof. R⁷ is also preferably a substituted alkyl wherein the preferred substituents are form ether linkages or contain sulfinic or sulfonic acid moieties. Other preferred substituents include sulfhydryl substituted alkyl substituents. Still other preferred substituents include CONR₂ wherein R is defined as above.

It is preferred that the indicated dotted line represents a double bond; however, compounds which contain a saturated [symbol] ring are also included within the scope of the invention.

Preferably, the mandatory substituent CA or CR⁸A is in the 3-position; regardless of which position this substituent occupies, the other position is CR¹, CR¹₂, NR⁶ or N. CR¹ is preferred. Preferred embodiments of R¹ include hydrogen, alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR², SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R¹ can be joined to form a fused, optionally substituted aromatic or nonaromatic, saturated or unsaturated ring which contains 3-8 members. Most preferably, R¹ is H, alkyl, such as methyl, most preferably, the ring labeled [symbol] contains a double bond and CR¹ is CH or C-alkyl. Other preferable forms of R¹ include H, alkyl, acyl, aryl, arylalkyl, heteroalkyl, heteroaryl, halo, OR, NR₂, SR, NRCOR, alkyl-OOR, RCO, COOR, and CN, wherein each R is independently H, alkyl, or aryl or heteroforms thereof.

While the position not occupied by CA is preferred to include CR¹, the position can also be N or NR⁶. While NR⁶ is less preferred (as in that case the ring labeled [symbol] would be saturated), if NR⁶ is present, preferred embodiments of R⁶ include H, or alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, or R₃Si wherein each R is independently H, alkyl, alkenyl or aryl or heteroforms thereof.

Preferably, CR⁸A or CA occupy position 3- and preferably Z² in that position is CA. However, if the [symbol] ring is saturated and R⁸ is present, preferred embodiments for R⁸ include H, halo, alkyl, alkenyl and the like. Preferably R⁸ is a relatively small substituent corresponding, for example, to H or lower alkyl 1-4C.

A is —Wᵢ—COXⱼY wherein Y is COR² or an isostere thereof and R² is a noninterfering substituent. Each of W and X is a spacer and may be, for example, optionally substituted alkyl, alkenyl, or alkynyl, each of i and j is 0 or 1. Preferably, W and X are unsubstituted. Preferably, j is 0 so that the two carbonyl groups are adjacent to each other. Preferably, also, i is 0 so that the proximal CO is adjacent the ring. However, compounds wherein the proximal CO is spaced from the ring can readily be prepared by selective reduction of an initially glyoxal substituted [symbol] ring. In the most preferred embodiments of the invention, the [symbol] / [symbol] ring system is an indole containing CA in position 3- and wherein A is COCR².

The noninterfering substituent represented by R², when R² is other than H, is a hydrocarbyl residue (1-20C) containing 0-5 heteroatoms selected from O, S and/or N or is an inorganic residue. Preferred are embodiments wherein R² is H, or is straight or branched chain alkyl, alkenyl, alkynyl, aryl, arylalkyl, heteroalkyl, heteroaryl, or heteroarylalkyl, each optionally substituted with halo, alkyl, heteroalkyl, SR, OR, NR², OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroatom-containing forms thereof, or wherein R² is OR, NR₂, SR, NRCONR₂, OCONR₂, or NRSO₂NR₂, wherein each R is independently H, alkyl, alkenyl or aryl or the heteroatom-containing forms thereof, and wherein two R attached to the same atom may form a 3-8 member ring and wherein said ring may further be substituted by alkyl, alkenyl, alkynyl, aryl, arylalkyl, heteroalkyl, heteroaryl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroatom-containing forms thereof wherein two R attached to the same atom may form a 3-8 member ring, optionally substituted as above defined.

Other preferred embodiments of R² are H, heteroarylalkyl, —NR₂, heteroaryl, —COOR, —NHRNR₂, heteroaryl-COOR, heteroaryloxy, —OR, heteroaryl-NR₂, —NROR and alkyl. Most preferably R² is isopropyl piperazinyl, methyl piperazinyl, dimethylamine, piperazinyl, isobutyl carboxylate, oxycarbonylethyl, morpholinyl, aminoethyldimethylamine, isobutyl carboxylate piperazinyl, oxypiperazinyl, ethylcarboxylate piperazinyl, methoxy, ethoxy, hydroxy, methyl, amine, aminoethyl pyrrolidinyl, aminopropanediol, piperidinyl, pyrrolidinyl-piperidinyl, or methyl piperidinyl.

Isosteres of COR² as represented by Y are defined as follows.

The isosteres have varying lipophilicity and may contribute to enhanced metabolic stability. Thus, Y, as shown, may be replaced by the isosteres in Table 1.

TABLE 1
Acid Isosteres

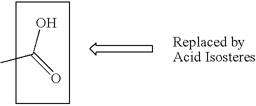

| Names of Groups | Chemical Structures | Substitution Groups (SG) |
|---|---|---|
| tetrazole | 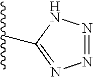 | n/a |
| 1,2,3-triazole | 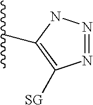 | H; SCH₃; COCH₃; Br; SOCH₃; SO₂CH₃; NO₂; CF₃; CN; COOMe |
| 1,2,4-triazole | 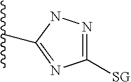 | H; SCH₃; COCH₃; Br; SOCH₃; SO₂CH₃; NO₂ |
| imidazole | 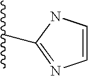 | H; SCH₃; COCH₃; Br; SOCH₃; SO₂CH₃; NO₂ |

Thus, isosteres include tetrazole, 1,2,3-triazole, 1,2,4-triazole and imidazole.

The compounds of formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

Compounds useful in the practice of the disclosed invention include, but are not limited to, the compounds shown in Table 2, below.

TABLE 2
Exemplary p38 Inhibitors

| Cpd. # | Mol. Structure |
|---|---|
| 1 | 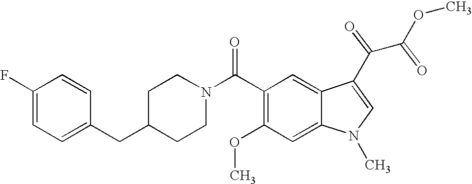 |
| 2 | 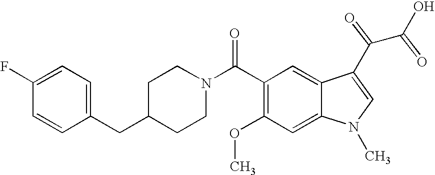 |
| 3 | 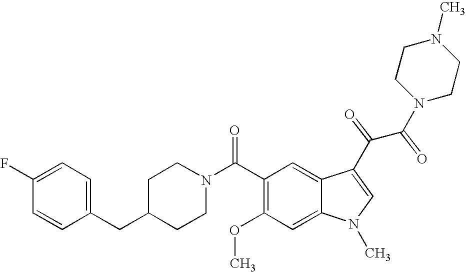 |
| 4 | 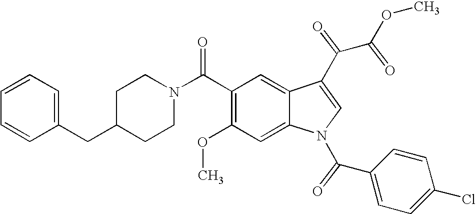 |
| 5 | 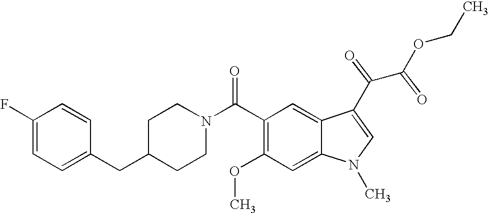 |
| 6 | 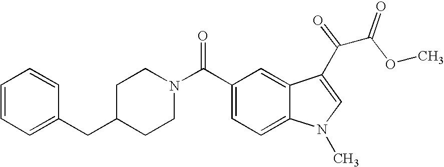 |
| 7 | 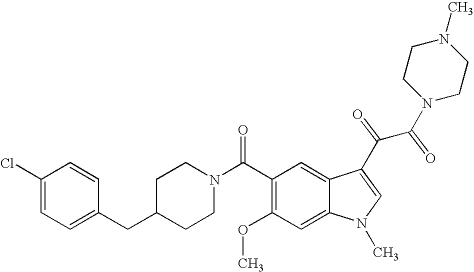 |
| 8 | 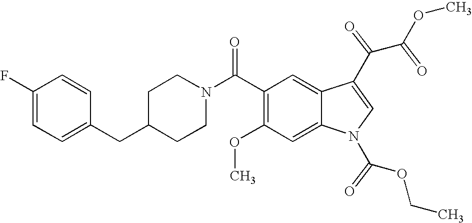 |
| 9 | 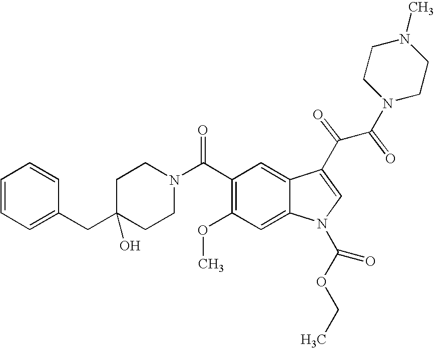 |
| 10 | 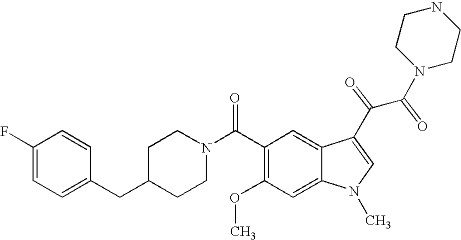 |
| 11 | 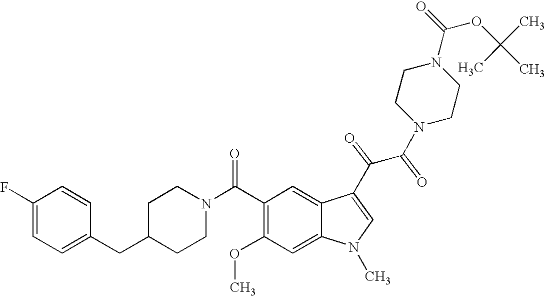 |
| 12 | 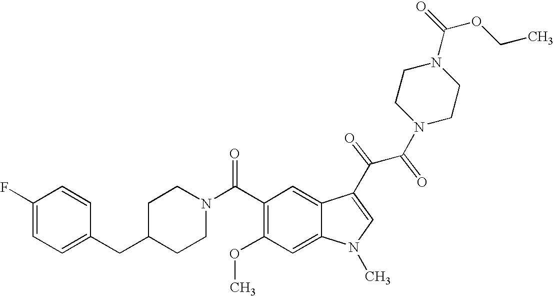 |
| 13 | 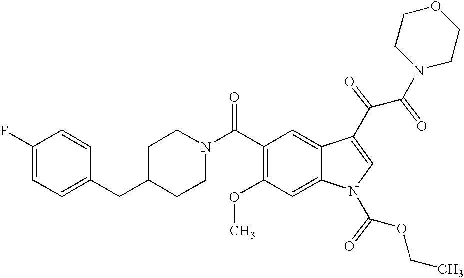 |
| 14 | 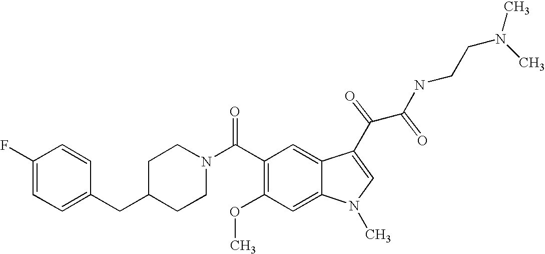 |
| 15 | 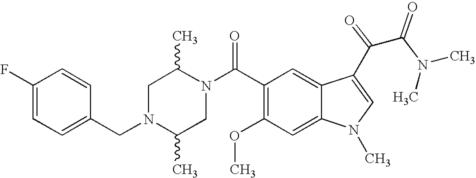 |
| 16 | 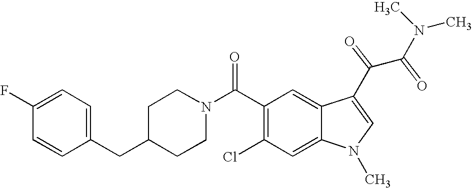 |
| 17 | 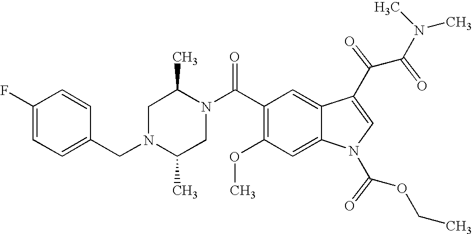 |
| 18 | 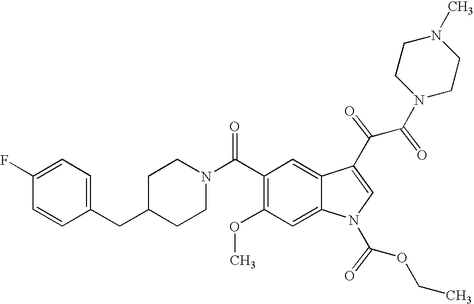 |
| 19 | 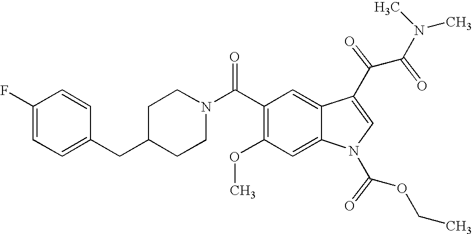 |
| 20 | 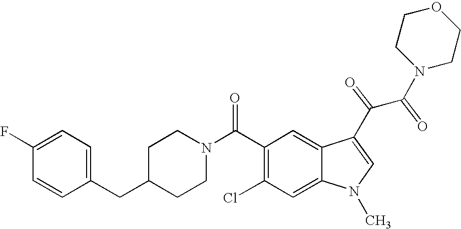 |
| 21 | 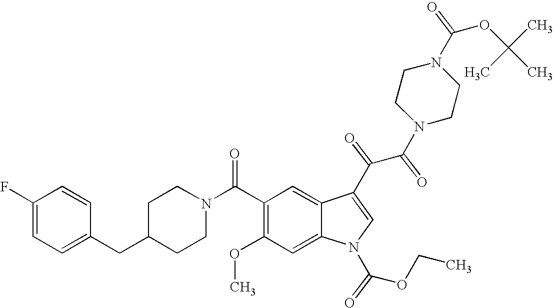 |
| 22 | 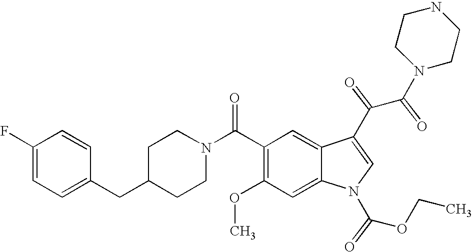 |
| 23 | 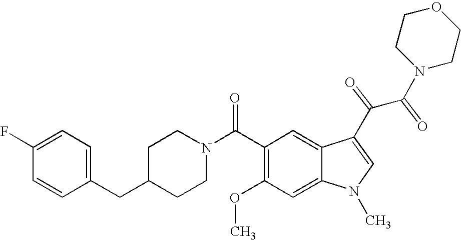 |
| 24 | 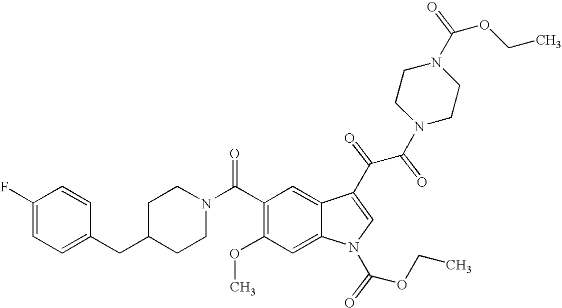 |
| 25 | 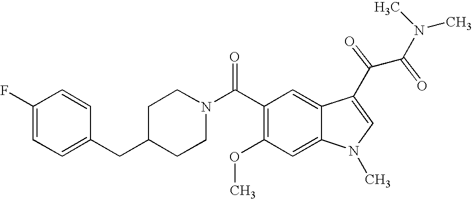 |
| 26 | 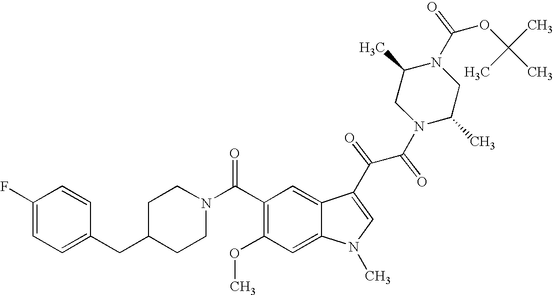 |
| 27 | 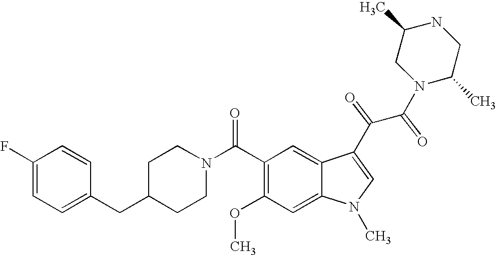 |
| 28 | 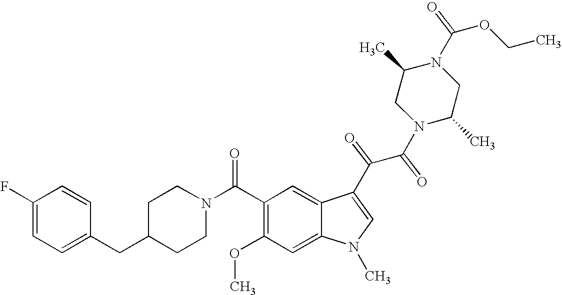 |
| 29 | 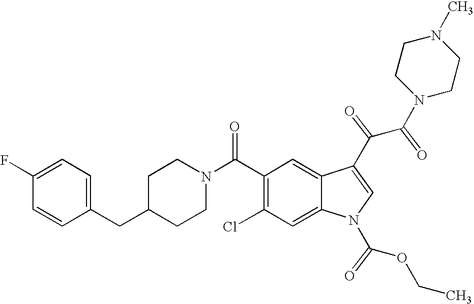 |
| 30 | 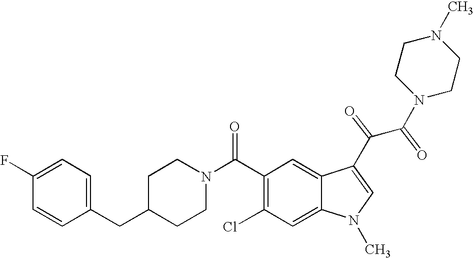 |
| 31 | 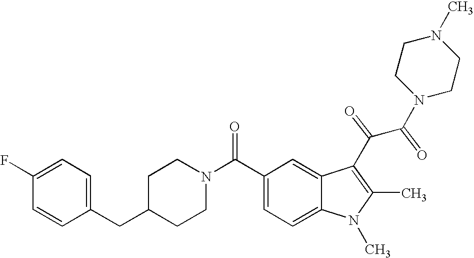 |
| 32 | 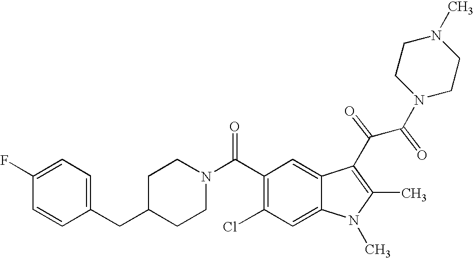 |
| 33 | 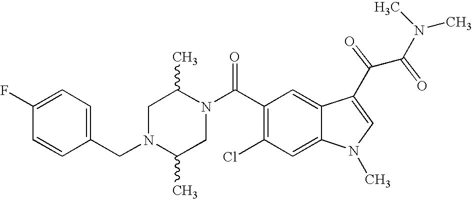 |
| 34 | 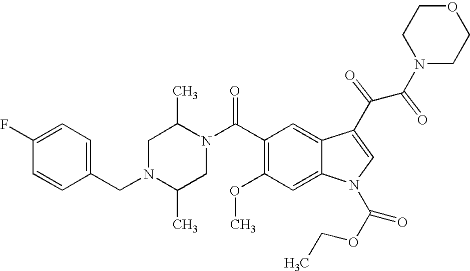 |
| 35 | 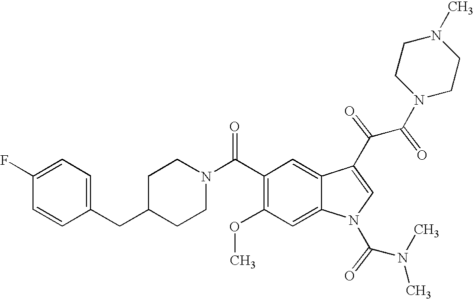 |
| 36 | 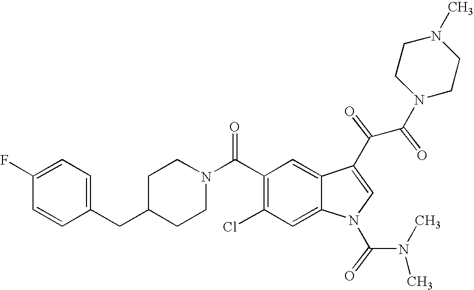 |
| 37 | 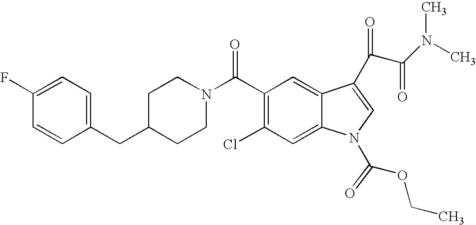 |
| 38 | 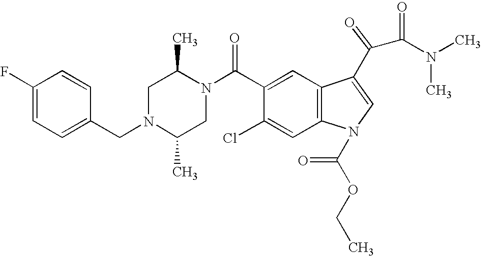 |
| 39 | 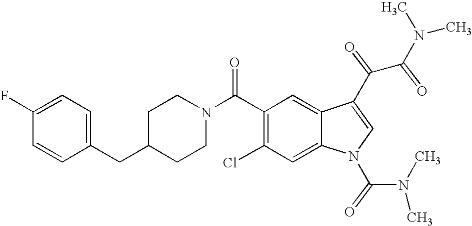 |
| 40 | 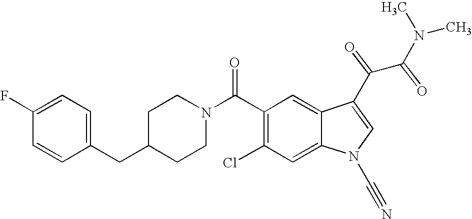 |
| 41 | 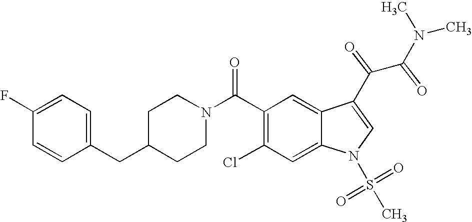 |
| 42 | 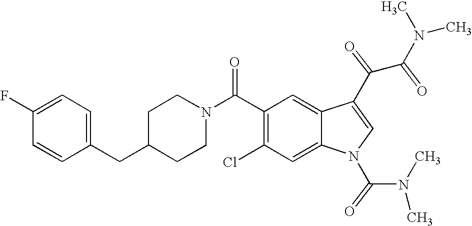 |
| 43 | 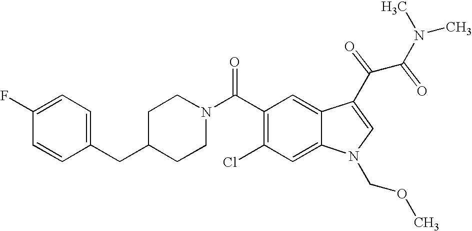 |
| 44 | 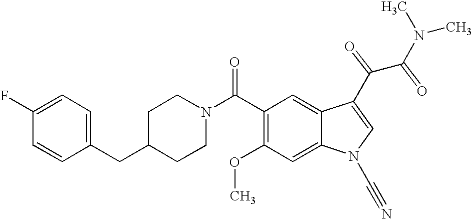 |
| 45 | 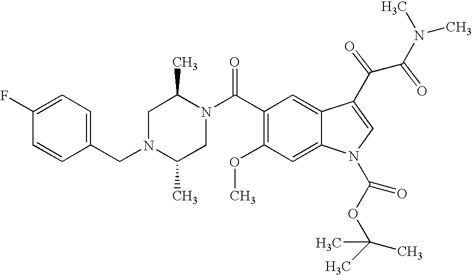 |
| 46 | 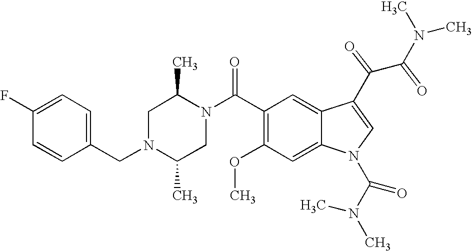 |
| 47 | 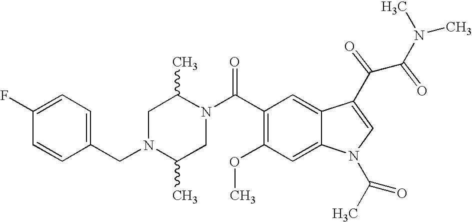 |
| 48 | 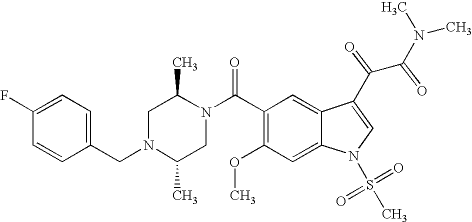 |
| 49 | 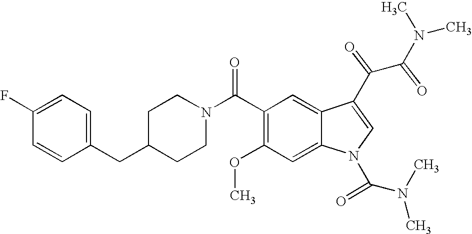 |
| 50 | 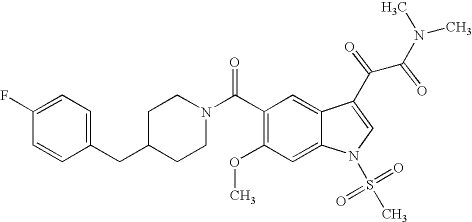 |
| 51 | 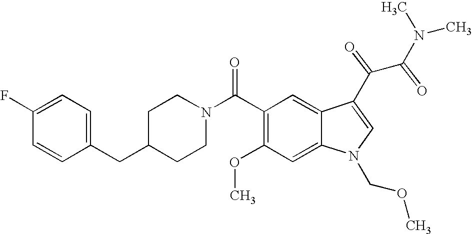 |
| 52 | 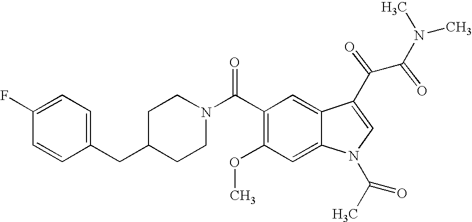 |
| 53 | 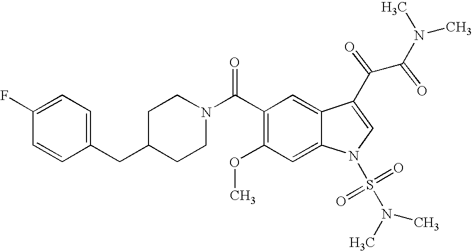 |
| 54 | 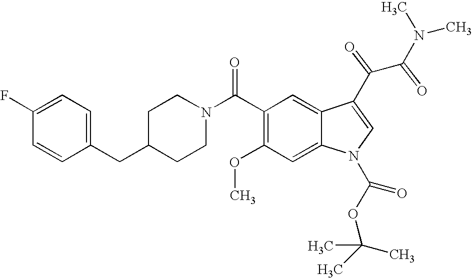 |
| 55 | 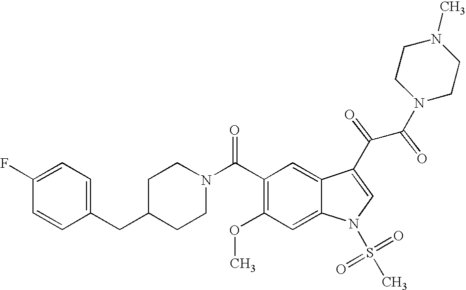 |
| 56 | 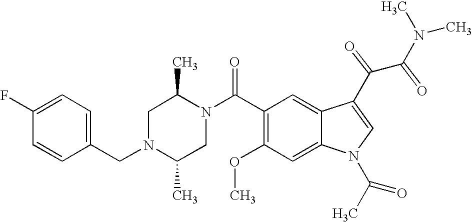 |
| 57 | 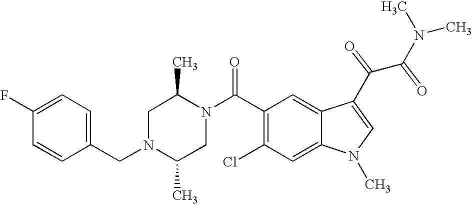 |
| 58 | 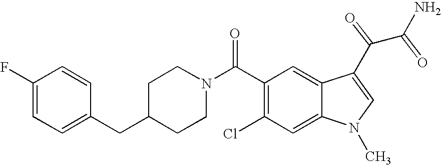 |
| 59 | 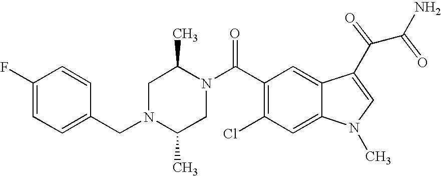 |
| 60 | 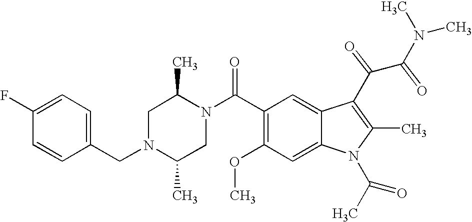 |
| 61 | 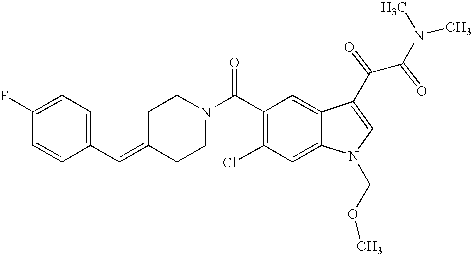 |
| 62 | 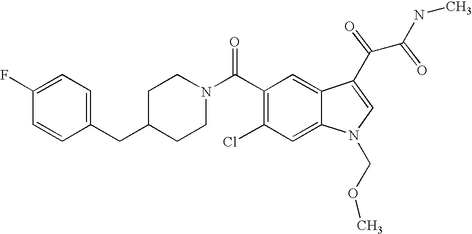 |
| 63 | 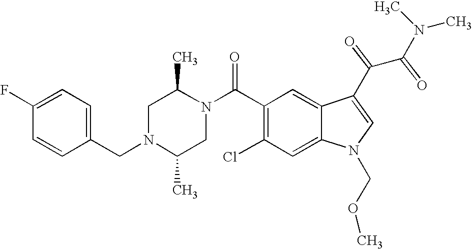 |
| 64 | 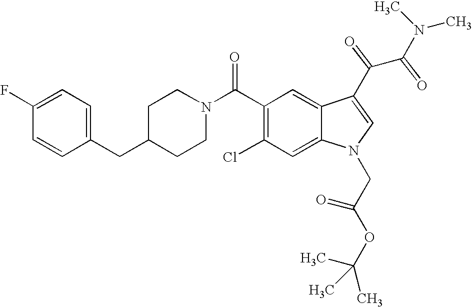 |
| 65 | 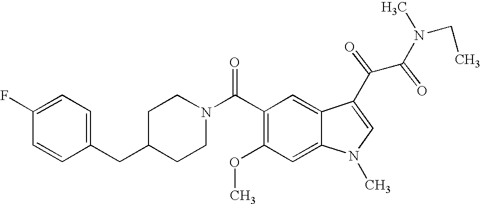 |
| 66 | 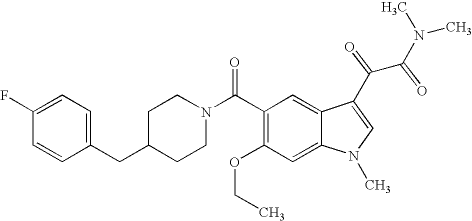 |
| 67 | 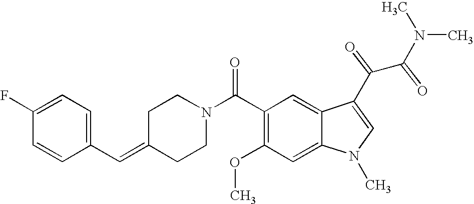 |
| 68 | 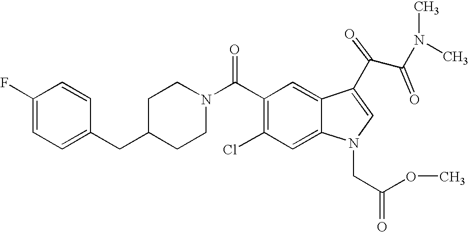 |
| 69 | 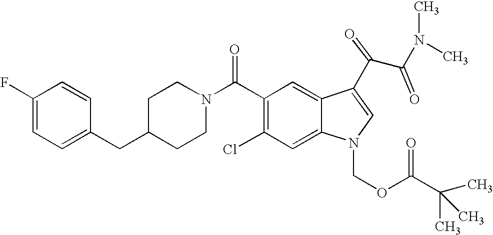 |
| 70 | 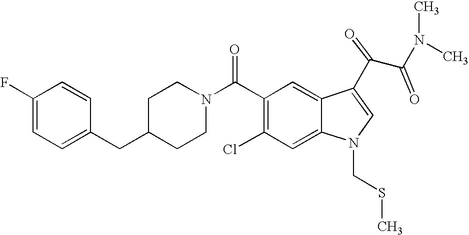 |
| 71 | 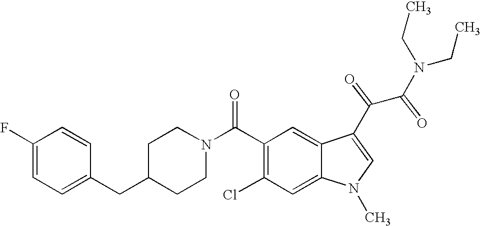 |
| 72 | 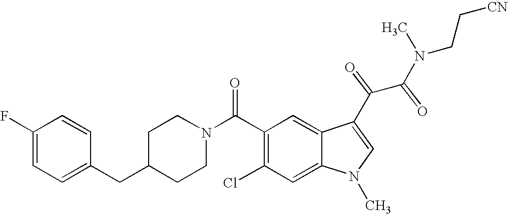 |
| 73 | 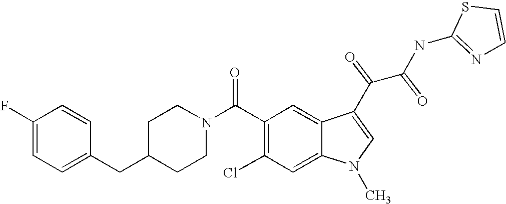 |
| 74 | 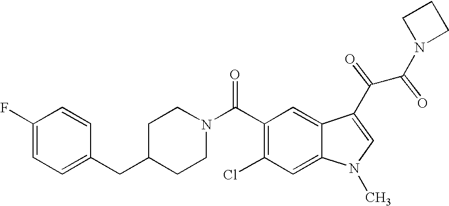 |
| 75 | 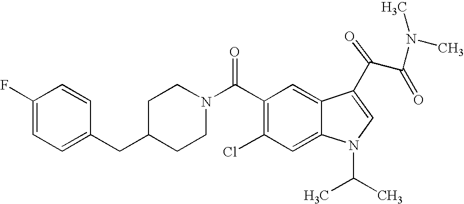 |
| 76 | 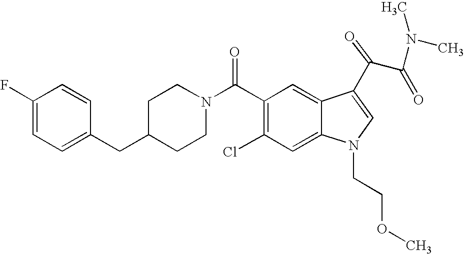 |
| 77 | 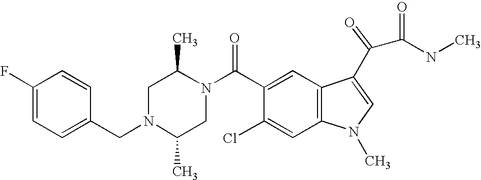 |
| 78 | 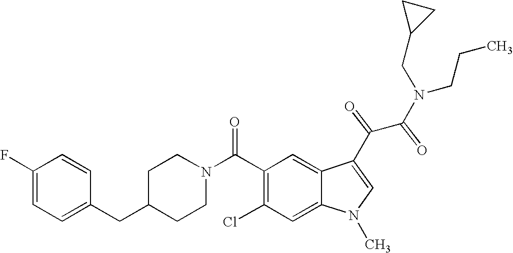 |
| 79 | 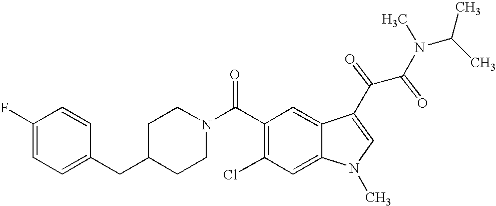 |
| 80 | 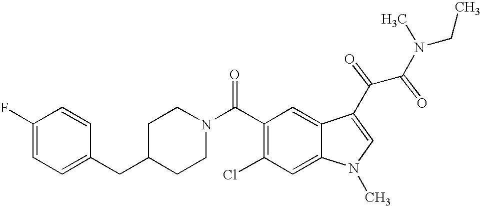 |
| 81 | 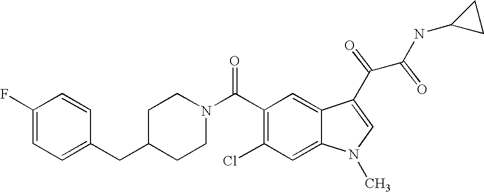 |
| 82 | 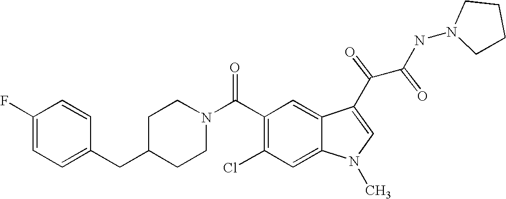 |
| 83 | 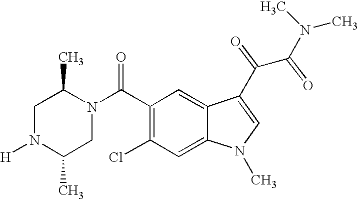 |
| 84 | 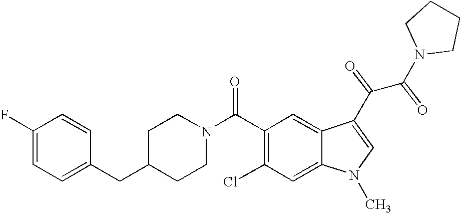 |
| 85 | 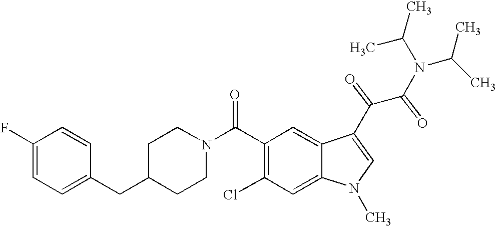 |
| 86 | 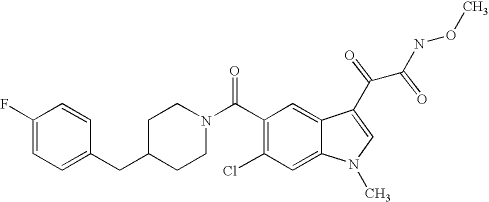 |
| 87 | 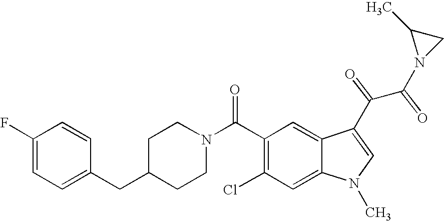 |
| 88 | 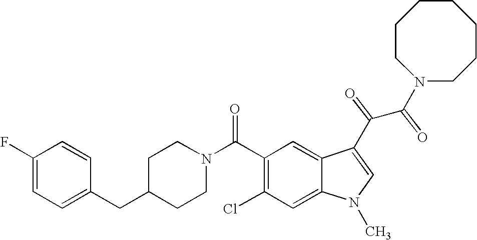 |
| 89 | 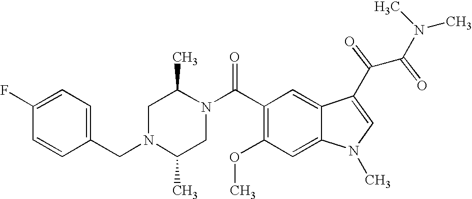 |
| 90 | 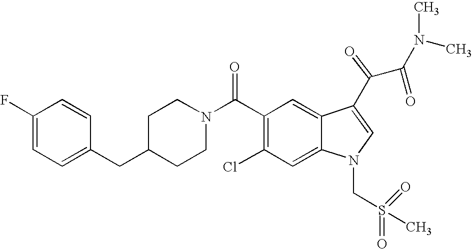 |
| 91 | 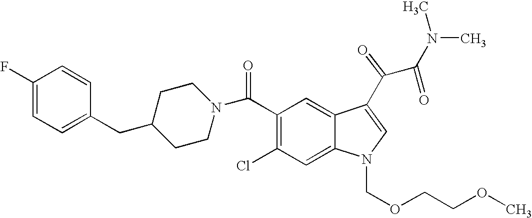 |
| 92 | 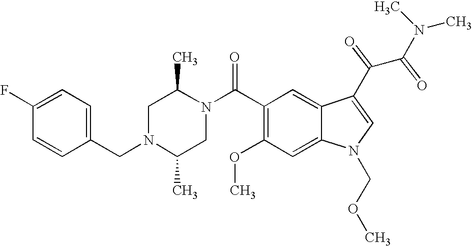 |
| 93 | 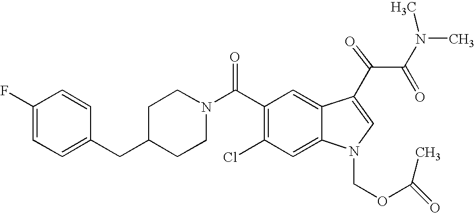 |
| 94 | 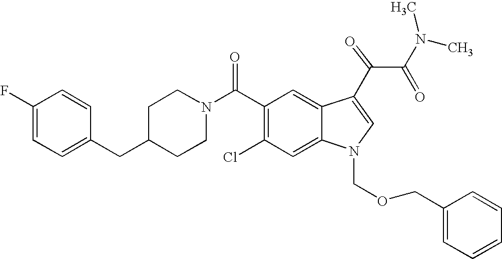 |
| 95 | 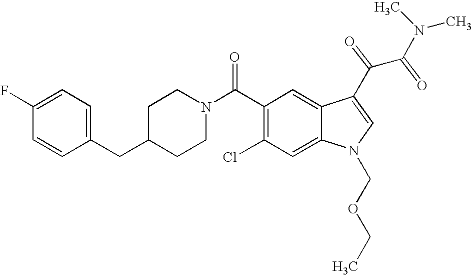 |
| 96 | 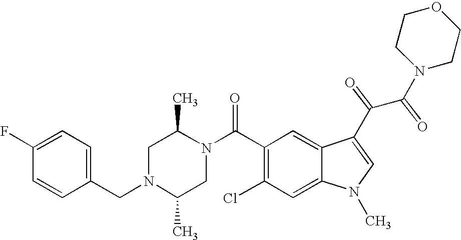 |
| 97 | 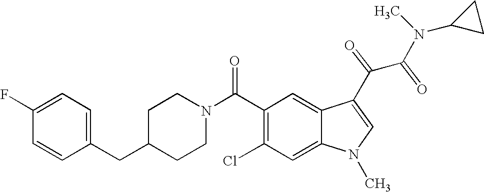 |
| 98 | 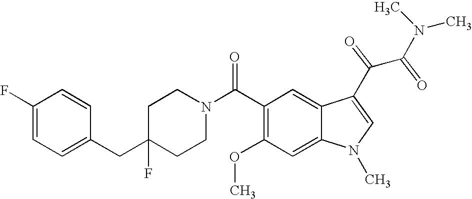 |
| 99 | 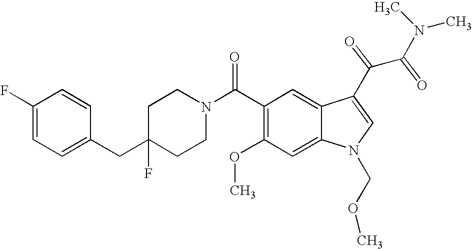 |
| 100 | 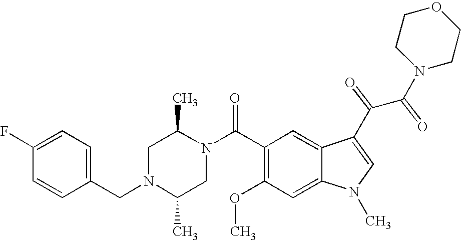 |
| 101 | 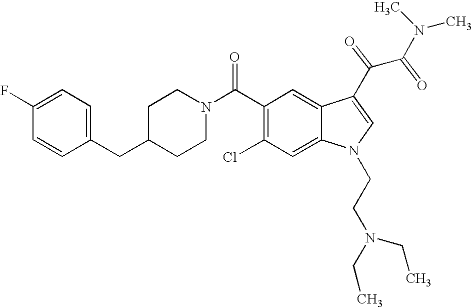 |
| 102 | 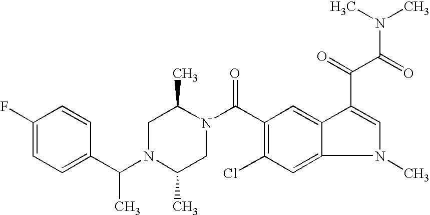 |
| 103 | 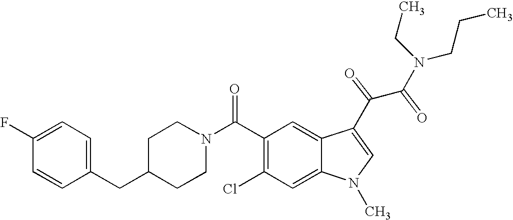 |
| 104 | 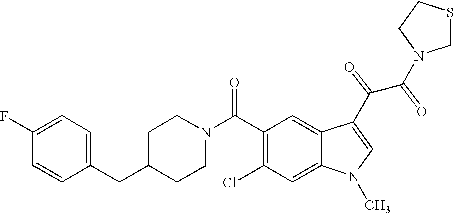 |
| 105 | 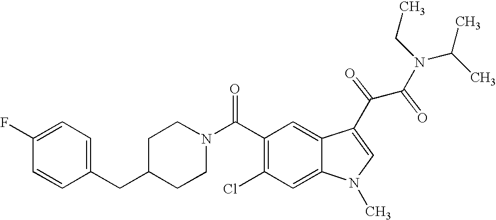 |
| 106 | 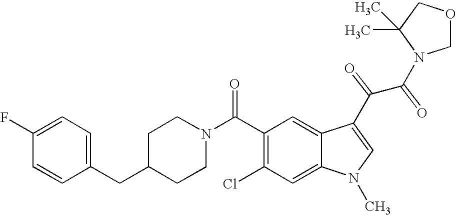 |
| 107 | 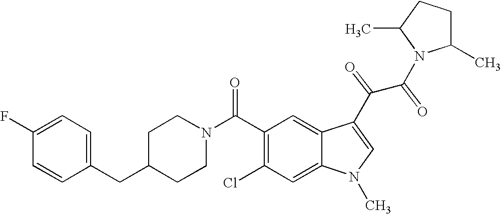 |
| 108 | 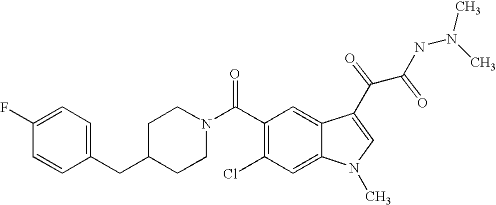 |
| 109 | 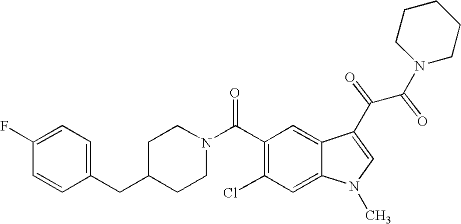 |
| 110 | 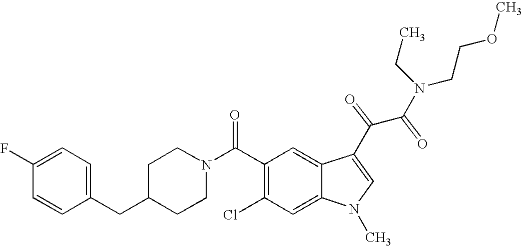 |
| 111 | 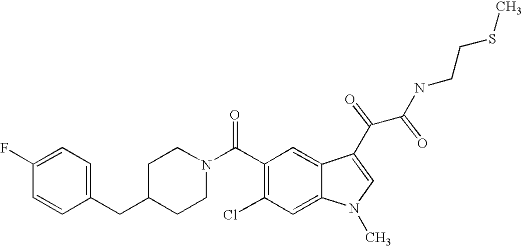 |
| 112 | 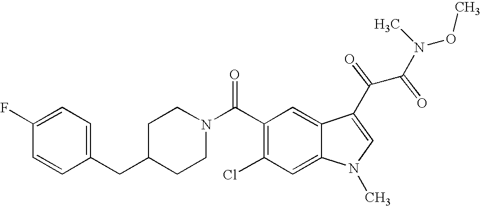 |
| 113 | 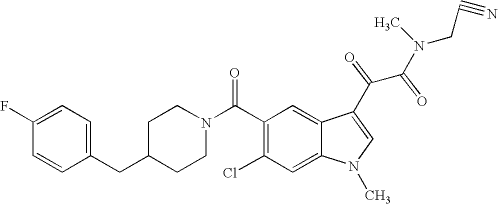 |
| 114 | 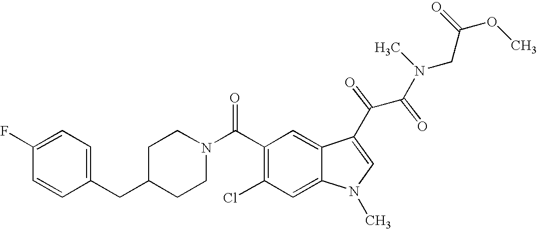 |
| 115 | 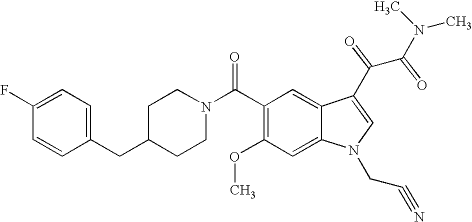 |
| 116 | 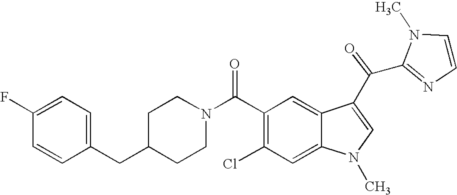 |
| 117 | 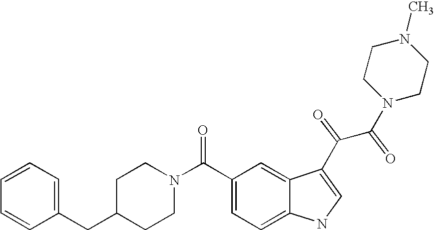 |
| 118 | 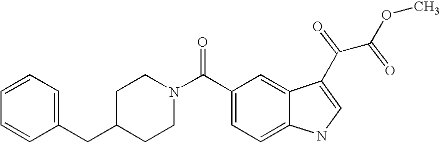 |
| 119 | 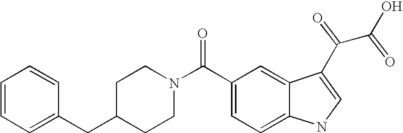 |
| 120 | 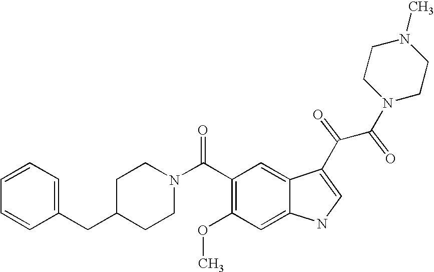 |
| 121 | 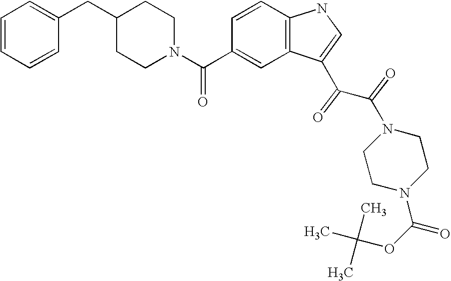 |
| 122 | 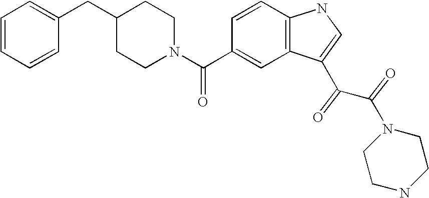 |
| 123 | 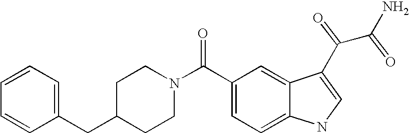 |
| 124 | 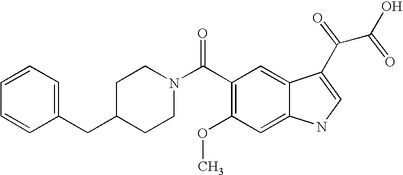 |
| 125 | 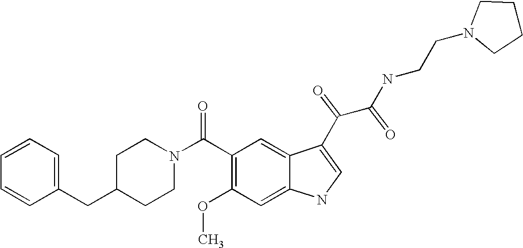 |
| 126 | 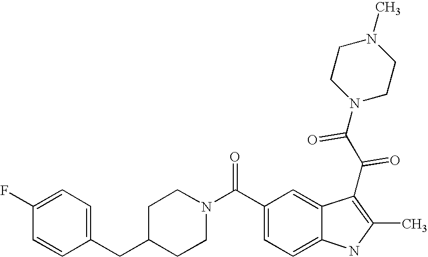 |
| 127 | 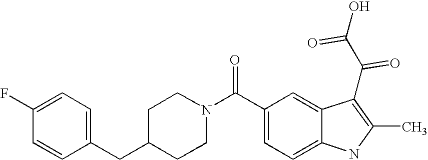 |
| 128 | 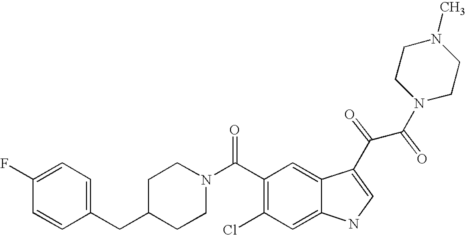 |
| 129 | 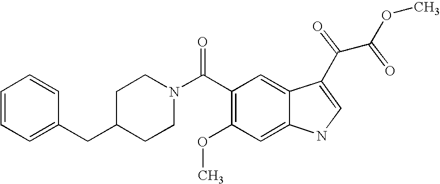 |
| 130 | 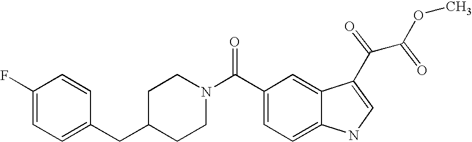 |
| 131 | 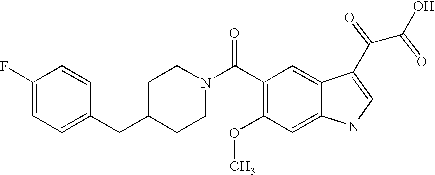 |
| 132 | 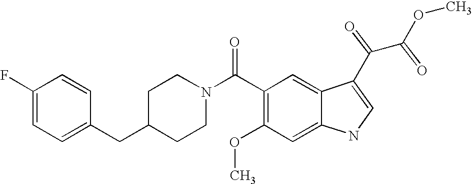 |
| 133 | 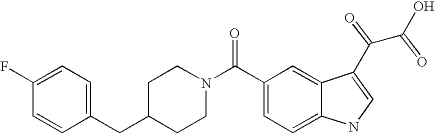 |
| 134 | 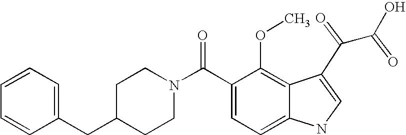 |
| 135 | 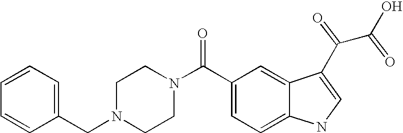 |
| 136 | 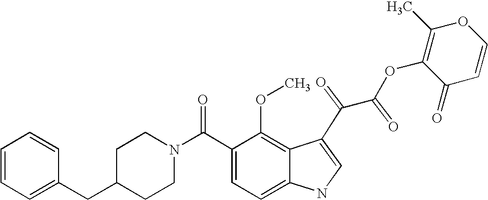 |
| 137 | 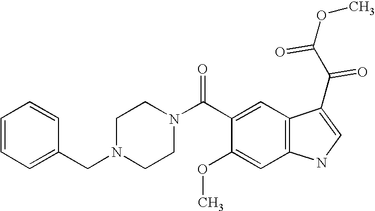 |
| 138 | 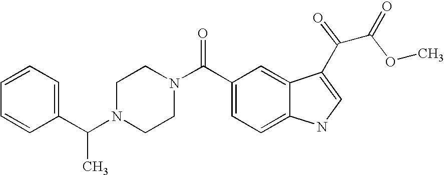 |
| 139 | 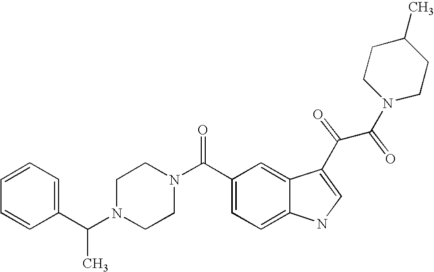 |
| 140 | 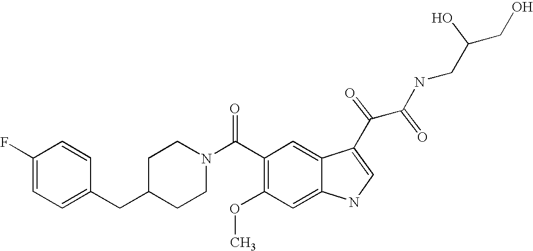 |
| 141 | 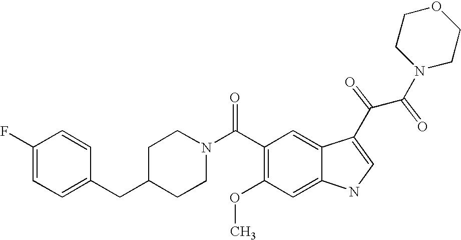 |
| 142 | 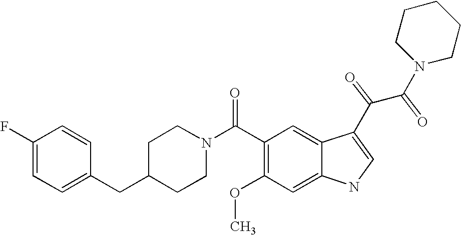 |
| 143 | 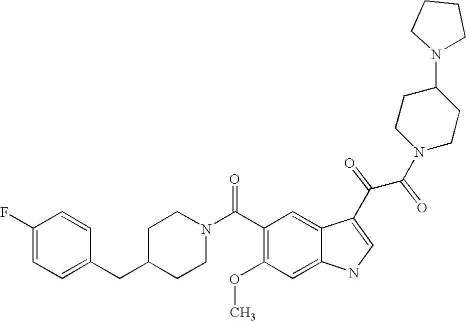 |
| 144 | 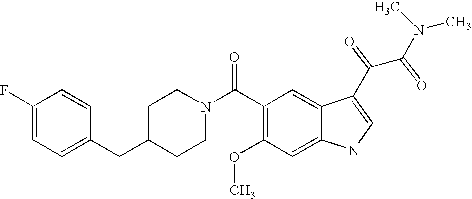 |
| 145 | 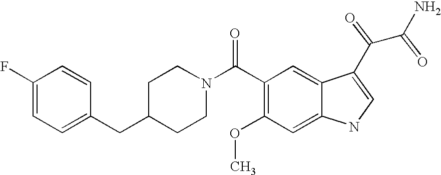 |
| 146 | 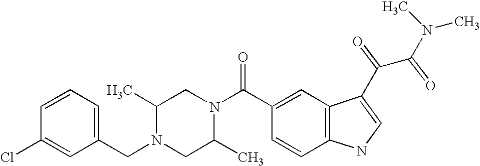 |
| 147 | 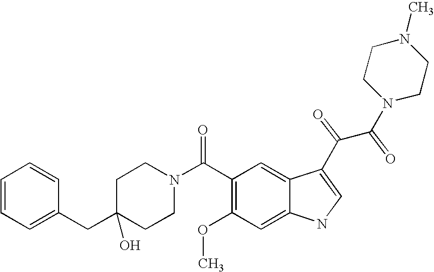 |
| 148 | 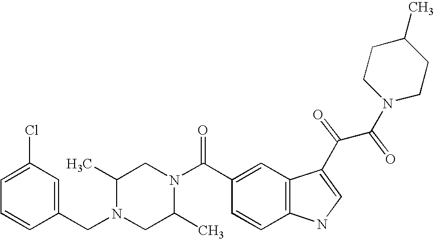 |
| 149 | 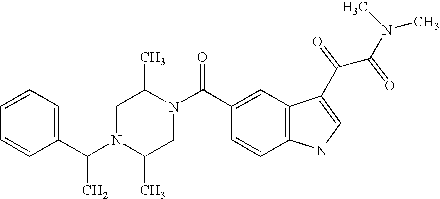 |
| 150 | 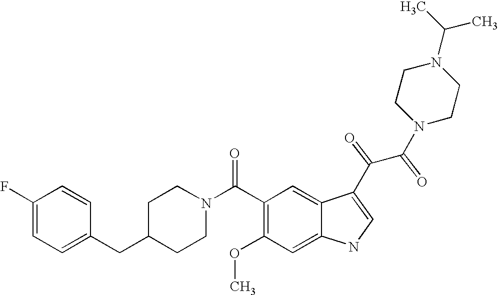 |
| 151 | 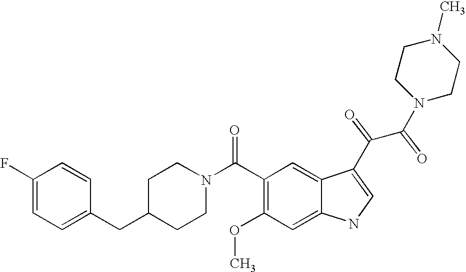 |
| 152 | 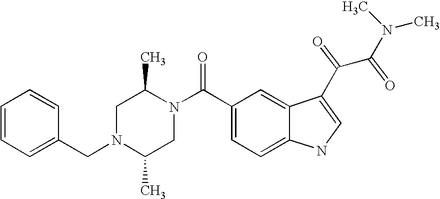 |
| 153 | 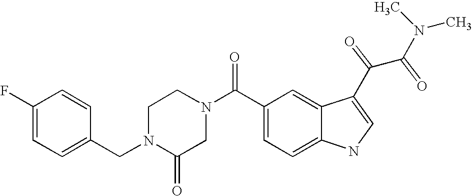 |
| 154 | 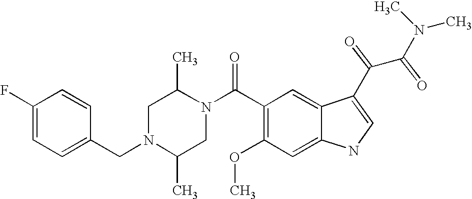 |
| 155 | 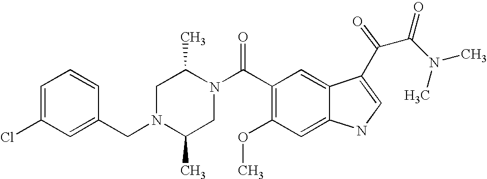 |
| 156 | 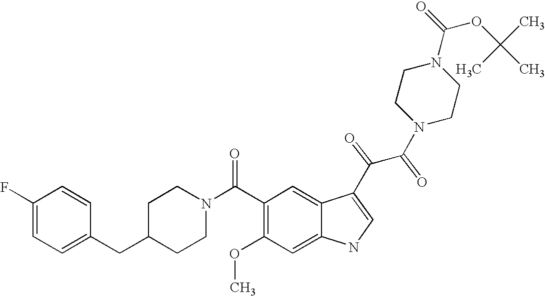 |
| 157 | 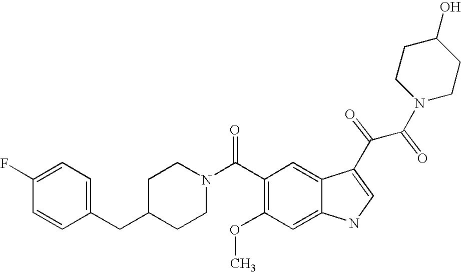 |
| 158 | 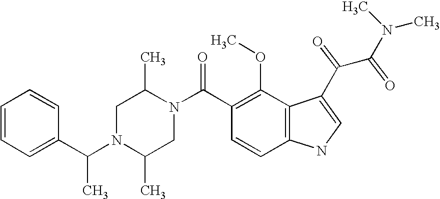 |
| 159 | 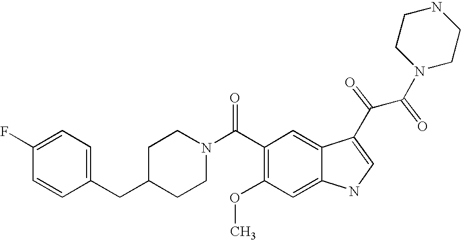 |
| 160 | 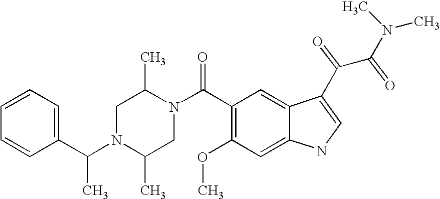 |
| 161 | 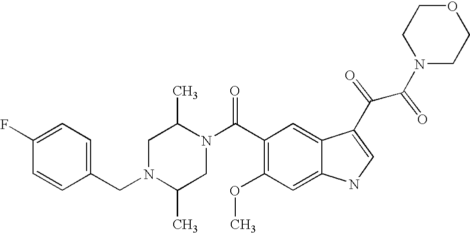 |
| 162 | 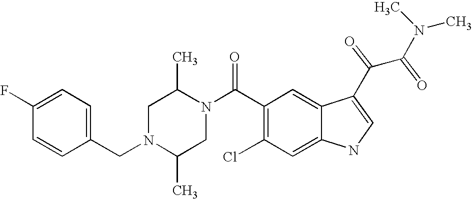 |
| 163 | 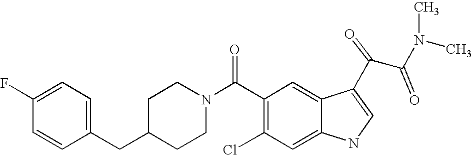 |
| 164 | 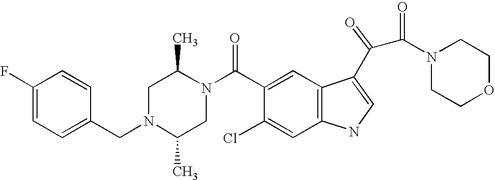 |
| 165 | 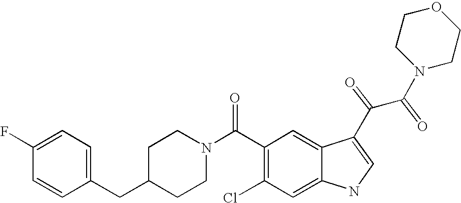 |
| 166 | 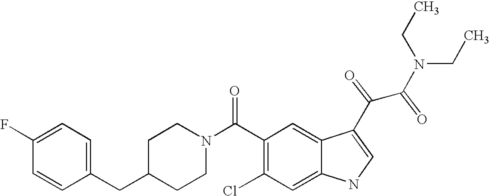 |
| 167 | 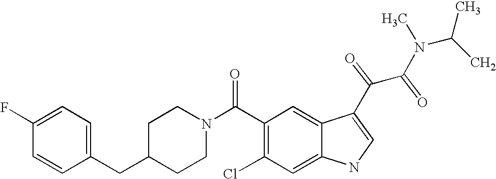 |
| 168 | 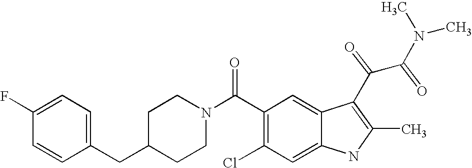 |
| 169 | 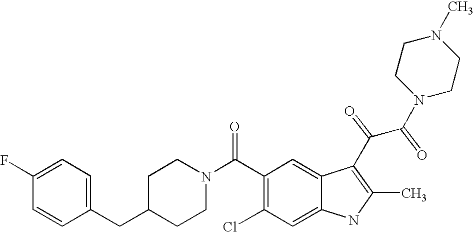 |
| 170 | 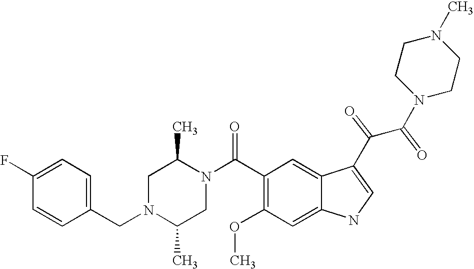 |
| 171 | 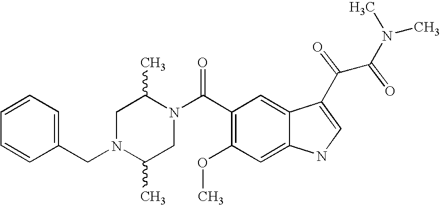 |
| 172 | 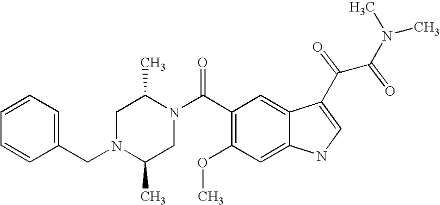 |
| 173 | 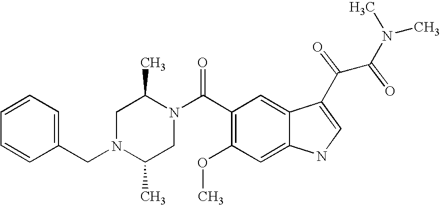 |
| 174 | 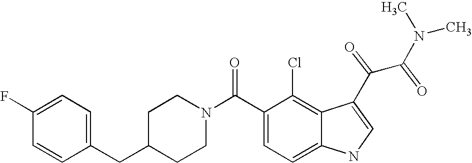 |
| 175 | 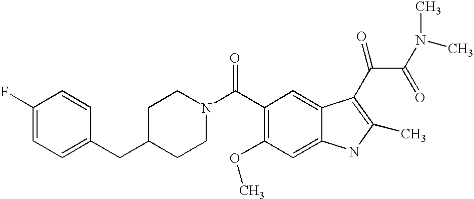 |
| 176 | 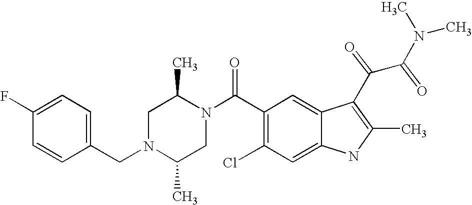 |
| 177 | 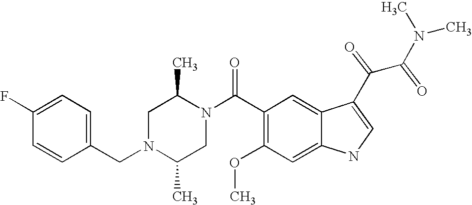 |
| 178 | 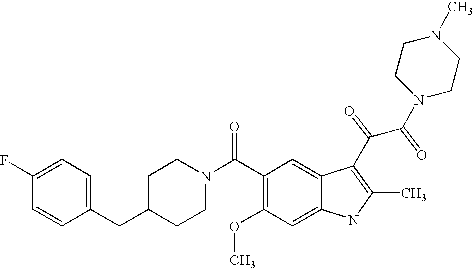 |
| 179 | 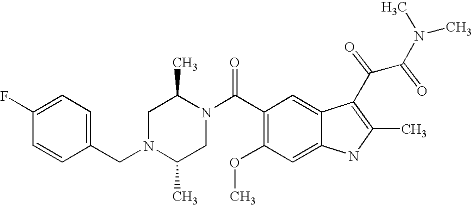 |
| 180 | 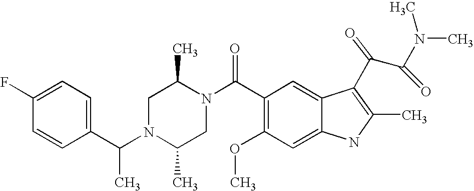 |
| 181 | 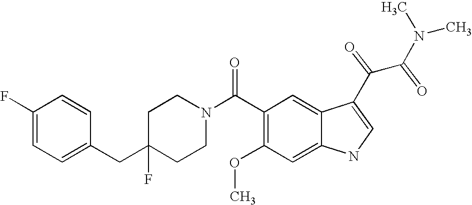 |

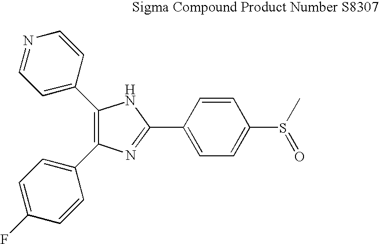

Additional compounds are described in published PCT applications WO 96/21452, WO 96/40143, WO 97/25046, WO 97/35856, WO 98/25619, WO 98/56377, WO 98/57966, WO 99/32110, WO 99/32121, WO 99/32463, WO 99/61440, WO 99/64400, WO 00/10563, WO 00/17204, WO 00/19824, WO 00/41698, WO 00/64422, WO 00/71535, WO 01/38324, WO 01/64679, WO 01/66539, and WO 01/66540, each of which is herein incorporated by reference in their entirety.

Further additional compounds useful in the practice of the present invention also include, but are not limited to, the compounds shown in Table 3, below.

TABLE 3

| | Citations, each of which is herein |
|---|---|
| Chemical Structure | incorporated by reference. |
| 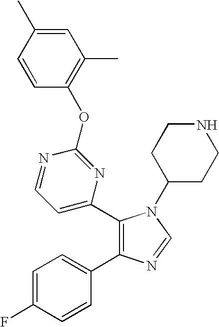 | WO-00166539, WO-00166540, WO-00164679, WO-00138324, WO-00064422, WO-00019824, WO-00010563, WO-09961440, WO-09932121, WO-09857966, WO-09856377, WO-09825619, WO-05756499, WO-09735856, WO-09725046, WO-09640143, WO-09621452; Gallagher, T. F., et. Al., Bioorg. Med. Chem. 5: 49 (1997); Adams, J. L., et al., Bioorg. Med. Chem. Lett. 8: 3111-3116 (1998) |
| 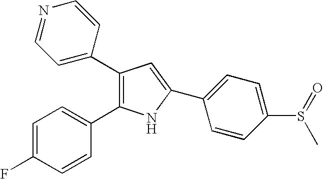 | De Laszlo, S. E., et. Al., Bioorg Med Chem Lett. 8: 2698 (1998) |
| 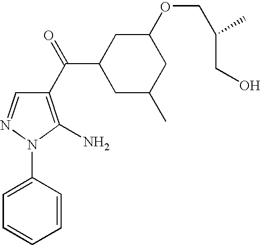 | WO-09957101; Poster presentation at the 5^{th} World Congress on Inflammation, Edinburgh, UK. (2001) |
| 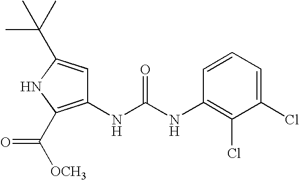 | WO-00041698, WO-09932110, WO-09932463 |
| 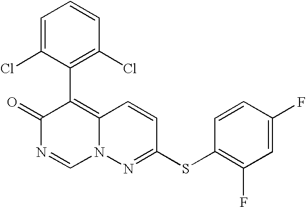 | WO-00017204, WO-09964400 |
| 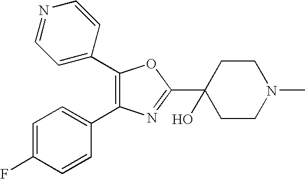 | Revesz. L., et. al., Bioorg Med Chem Left. 10: 1261 (2000) |
| 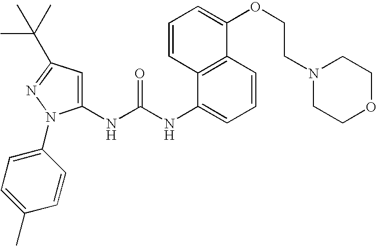 | WO-00207772 |
| 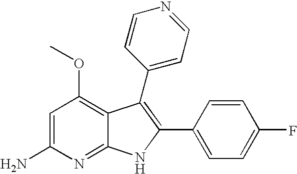 | Fijen, J. W., et al., Clin. Exp. Immunol. 124: 16-20 (2001); Wadsworth, S. A., et. al., J. Pharmacol. Expt. Therapeut. 291: 680 (1999) |
| 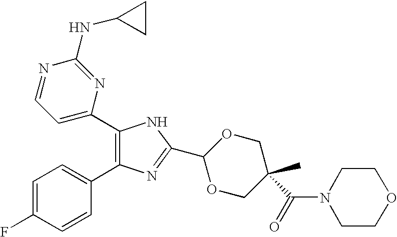 | Collis, A. J., et al.. Bioorg. Med Chem. Lett. 11: 693-696 (2001); McLay, L. M., et al., Bioorg Med Chem 9: 537—554 (2001) |
| 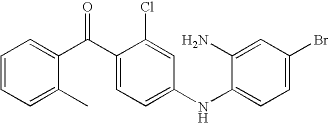 | WO-00110865, WO-00105749 |

Additional guidance regarding p38 MAPK inhibitory compounds is found in U.S. patent application Ser. Nos. 09/575,060, 10/157,048, 10/146,703, 10/156,997, and 10/156,996, all of which are hereby incorporated by reference in their entirety. The compounds described above are provided for guidance and exemplary purposes only. It should be understood that any modulator of p38 MAP kinase is useful for the invention provided that it exhibits adequate activity relative to the targeted protein.

Utility and Administration

The methods and compositions of the invention are successful to treat or ameliorate multiple myeloma in humans.

As used herein, “treat” or “treatment” include effecting postponement of development of undesirable conditions and/or reduction in the severity of such symptoms that will or are expected to develop. Treatment includes ameliorating existing symptoms, preventing additional symptoms, ameliorating or preventing the underlying metabolic causes of symptoms, preventing the severity of the condition or reversing the condition, at least partially. Thus, the terms denote that a beneficial result has been conferred on a subject with multiple myeloma.

Treatment generally comprises “administering” to a subject a compound which includes providing the subject compound in a therapeutically effective amount. “Therapeutically effective amount” means the amount of the compound that will treat multiple myeloma by eliciting a favorable response in a cell, tissue, organ, system, in a human. The response may be preventive or therapeutic. The administering may be of the compound per se in a pharmaceutically acceptable composition, or this composition may include combinations with other active ingredients that are suitable to the treatment of this condition. The compounds may be administered in a prodrug form.

The manner of administration and formulation of the compounds useful in the invention and their related compounds will depend on the composition of the compound, the nature of the condition, the severity of the condition, the particular subject to be treated, and the judgment of the practitioner; formulation will also depend on mode of administration. For example, if the compounds are “small molecules,” they might be conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like. This method is preferred if the subject can tolerate oral administration.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Intravenous administration is preferred for acute conditions; generally in these circumstances, the subject will be hospitalized. The intravenous route avoids any problems with inability to absorb the orally administered drug.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

Thus, the compounds useful in the method of the invention may be administered systemically or locally. For systemic use, the compounds are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Alternatively, the compounds may be administered in a cyclical manner (administration of compound; followed by no administration; followed by administration of compound, and the like). Treatment will continue until the desired outcome is achieved. In general, pharmaceutical formulations will include an active ingredient in combination with a pharmaceutically acceptable vehicle, such as saline, buffered saline, 5% dextrose in water, borate-buffered saline containing trace metals or the like. Formulations may further include one or more excipients, preservatives, solubilizers, buffering agents, albumin to prevent protein loss on vial surfaces, lubricants, fillers, stabilizers, etc.

Pharmaceutical compositions can be in the form of sterile, non-pyrogenic liquid solutions or suspensions, coated capsules, suppositories, lyophilized powders, transdermal patches or other forms known in the art.

Biodegradable films or matrices may be used in the invention methods. These include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials, to provide desired mechanical, cosmetic or tissue or matrix interface properties.

Alternative methods for delivery may include osmotic minipumps; sustained release matrix materials such as electrically charged dextran beads; collagen-based delivery systems, for example; methylcellulose gel systems; alginate-based systems, and the like.

Aqueous suspensions may contain the active ingredient in admixture with pharmacologically acceptable excipients, comprising suspending agents, such as methyl cellulose; and wetting agents, such as lecithin, lysolecithin or long-chain fatty alcohols. The said aqueous suspensions may also contain preservatives, coloring agents, flavoring agents, sweetening agents and the like in accordance with industry standards.

Preparations for topical and local application comprise aerosol sprays, lotions, gels and ointments in pharmaceutically appropriate vehicles which may comprise lower aliphatic alcohols, polyglycols such as glycerol, polyethylene glycol, esters of fatty acids, oils and fats, and silicones. The preparations may further comprise antioxidants, such as ascorbic acid or tocopherol, and preservatives, such as p-hydroxybenzoic acid esters.

Parenteral preparations comprise particularly sterile or sterilized products. Injectable compositions may be provided containing the active compound and any of the well known injectable carriers. These may contain salts for regulating the osmotic pressure.

Liposomes may also be used as a vehicle, prepared from any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol and the like. Synthetic phospholipids may also be used.

The dosages of the compounds of the invention will depend on a number of factors which will vary from subject to subject. However, it is believed that generally, the daily dosage in humans (average weight of 70 kg) will range between 30 mg and 500 mg, preferably between 45 mg and 400 mg, more preferably between 50 mg and 300 mg per day. The dose regimen will vary, however, depending on the compound and formulation selected, the condition of the subject being treated and the judgment of the practitioner. Optimization of dosage, formulation and regimen is routine for practitioners of the art.

The following examples are intended to illustrate but not to limit the invention.

### EXAMPLES

The following examples describe experiments to evaluate the effectiveness of p38 MAPK inhibitors as a treatment for multiple myeloma in a patient in need thereof. Table 2 lists a number of compounds that generally exhibit p38 MAPK activity, preferred embodiments exhibit a relative IC₅₀ value of less than 5 nM in an assay similar to the phosphorylation assay disclosed above (see Kumar). The compounds listed in Table 2 exemplify the compounds generically disclosed herein. Moreover, the data discussed below is representative of the genus of p38 MAPK inhibitors disclosed herein. The results discussed below are thought to be obtainable using any of the p38 MAPK inhibitors disclosed herein. As such, the data provided demonstrates that the genus of p38MAPK inhibitor compounds disclosed herein are useful in the disclosed methods of treating multiple myeloma. The Sigma-Aldrich® under product number S8307 compound is 4-(4-Fluorophenyl)-2-(4-methylsulfinylphenyl)-5-(4-pyridyl)-1H-imidazole, which is known in the literature as a p38 MAPK modulator and commercial available. This compound is available as a positive control in a p38 MAPK inhibition assay.

### Example 1

### p38 Activation in Multiple Myeloma

p38 MAPK is activated via dual phosphorylation by MEKK3 and/or MEKK6. The activated form of p38α MAPK is found in untreated and TNFα-activated MM cells, and in BMSC, using p38α phospho-specific immunodetection. BMSC are obtained from seven different donors (three normal healthy individuals and four MM patients). In the experiments reported here, no differences between BMSCs obtained from patients and healthy individuals were noted. A variety of widely used MM cell lines were also used in the studies described below.

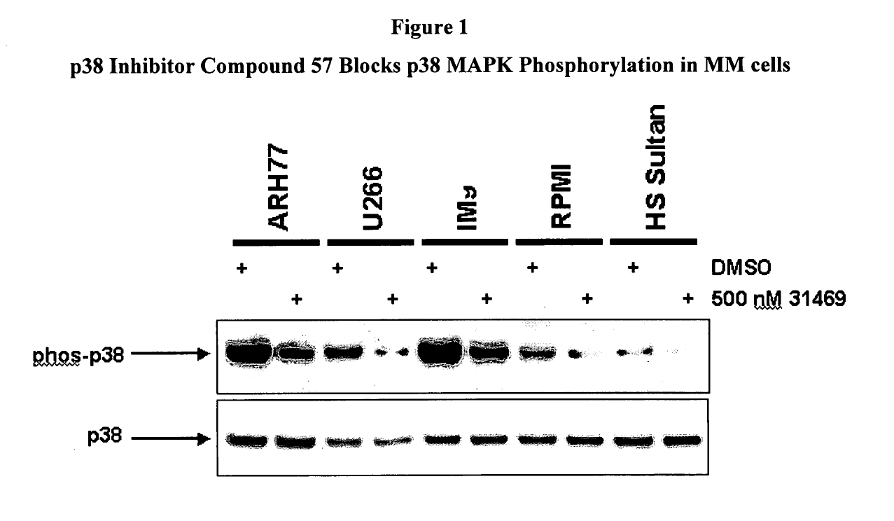

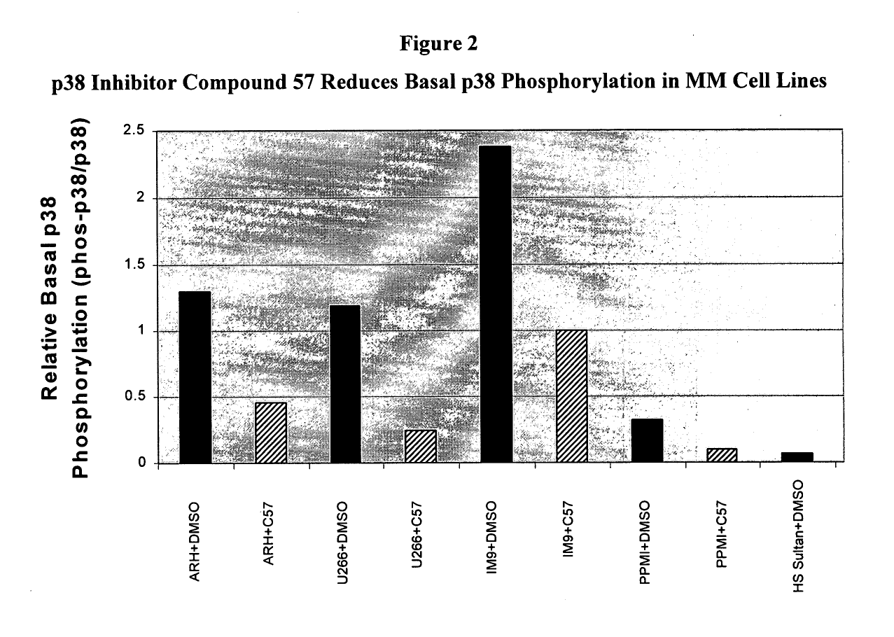

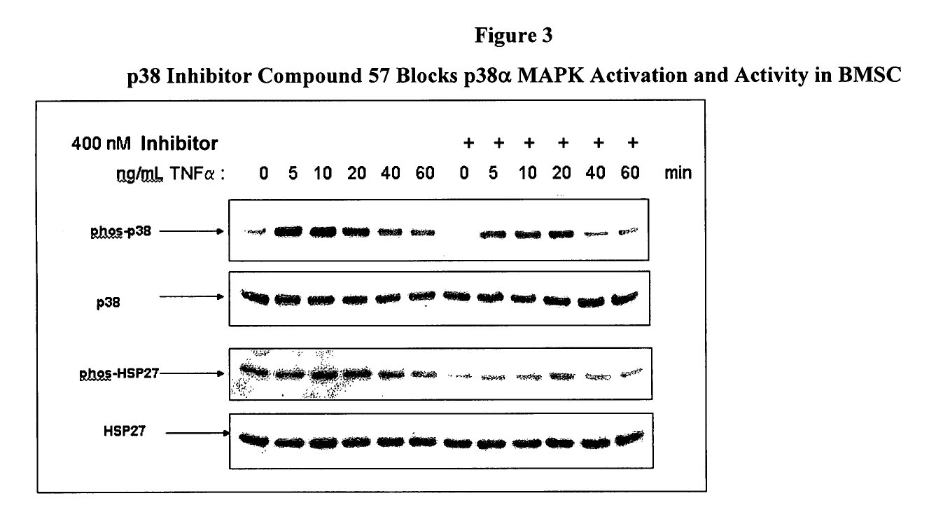

The phosphorylation of p38α MAPK in MM cells was substantially suppressed by the MAPK inhibitor Compound 57 shown in Table 2 (see FIG. 1 and FIG. 2), while the phosphorylation of p38α MAPK in BMSC was partially suppressed (FIG. 3). This inhibitor blocked activity of p38 MAPK, but not the direct activation of the p38 MAPK enzyme or the activity of kinases upstream of p38 MAPK (e.g., MKK3 and MKK6); therefore this cellular effect is presumed to result from disruption of a feedback loop involving p38 MAPK kinase activity. As expected, p38 MAPK activity was fully suppressed by the p38 MAPK inhibitor, shown by immunodetection of p38 MAPK kinase target HSP-27. The Compound 57 blocked phosphorylation of HSP-27 completely in MM cells and in BMSC.

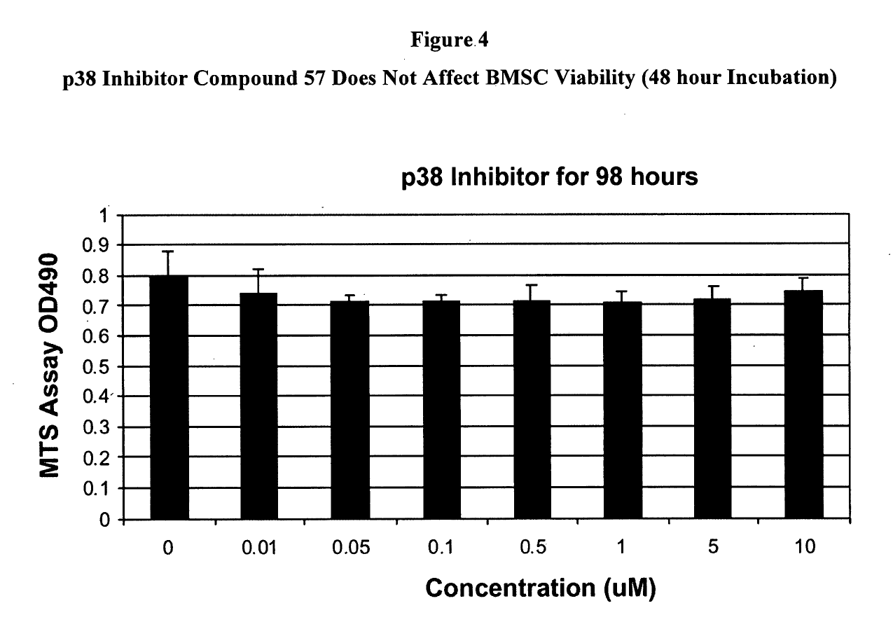

Neither p38 MAPK inhibition nor high concentrations of p38 MAPK inhibitor (tested up to 50-fold excess of active concentration) affected BMSC viability (FIG. 4). Viability was measured using a standard enzymatic assay of respiratory activity, MTS (3-(4,5-dimethylthiazol-2-yl)-5-(3-carboxymethoxyphenyl)-2-(4-sulfophenyl)-2H-tetrazolium). Results obtained with the p38 MAPK inhibitor are in agreement with published results using a different p38α MAPK inhibitor.

### Example 2

### Receptor Activator of Nuclear Factor-κB Ligand (RANKL)

RANKL is a powerful inducer of osteoclast differentiation and activation. This transcription factor is highly activated in MM cells and is activated by p38α MAPK. For this reason, experimental therapeutic strategies for MM bone lesions and other osteolytic diseases are focusing on inhibition of RANK signaling, including activation of NFκK. This transcription factor is highly activated in MM cells, and is activated by p38 MAPK. NFκK inhibition is believed to be a major mechanism in several demonstrated therapies for MM.

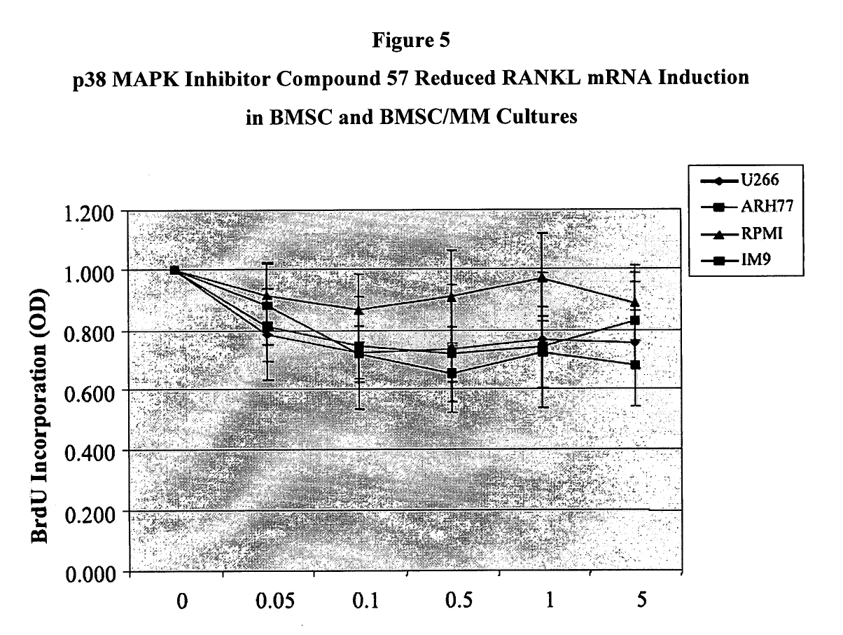

RANKL mRNA is powerfully induced by TGFβ and IL-1 in BMSC and MM/BMSC cultures. Exposure to 100 nM p38 MAPK inhibitors Compound 57 or the hydrochloride salt thereof blocked this induction, as well as basal expression of RANKL mRNA (FIG. 5). In addition, 100 nM of these p38 MAPK inhibitors blocks the weaker induction of RANKL by IL-6 in BMSC.

### Example 3

### p38 MAPK Inhibition in the Presence of Chemotherapeutic Agents and Potential Effects on MM Cells

p38 MAPK activity is necessary for production of factors such as IL-6, and therefore contributes to MM cell survival, even in the presence of chemotherapeutic agents. For example, a p38-mediated stress response in MM cells may invoke protective mechanisms, and in BMSC may induce secretion of MM survival factors such as IL-6. In fact, the unusual chemoresistance of MM cells compared to other B cell malignancies is thought to be due in part to IL-6-mediated support of MM cells.

Inhibition of p38 MAPK enhances the effects of conventional MM chemotherapeutic treatment and may function through one or both of two mechanisms: 1) by down-regulating the activity of one or more of these various factors; 2) through an overlap of mechanisms of action with some of the newer therapeutic agents. Proteasome inhibitors such as VELCADE block NFκK (a transcription factor central to inflammatory responses that is activated by p38 MAPK activity), and this may be at least partly responsible for their therapeutic benefit. Furthermore, the therapeutic effects of VELCADE, REVIMID, and thalidomide are believed to be due, in part, to decreased cytokine levels, including TNFα and VEGF, both of which are reduced by p38 MAPK inhibition.

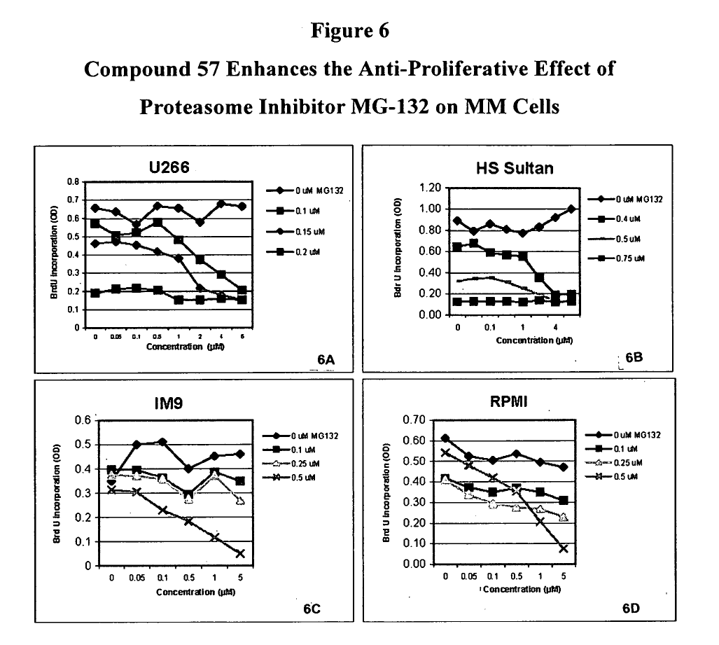

Co-exposure to the p38 MAPK inhibitor Compound 57 listed in Table 2 enhanced the reduction of MM cell viability and proliferation caused by exposure to proteasome inhibitor MG-132. MG-132 is a competitive inhibitor at the chymotryptic site of the proteasome, as is bortezomib. Note that IM9 MM cells were not sensitive to proteasome inhibitor MG-132 alone, even at doses able to block completely proliferation of U266 or HS Sultan MM cells. However, in the presence of the p38 MAPK inhibitor, IM9 proliferation is reduced or entirely blocked in a manner related to the dose of both MG-132 and of the p38 MAPK inhibitor. These interactions show the benefits of co-treatment regimes in MM. Since dose limiting toxicities are observed with many treatments, the ability to achieve equivalent efficacy at lower doses, or to allow tolerance of a higher dose in order to increase the response further, shows the clear potential for a therapeutic benefit of using p38 MAPK inhibition in treating MM. Co-exposure of MM cells to Compound 57 also enhances the ability of proteasome inhibitor MG-132 to reduce MM cell proliferation (FIG. 6).

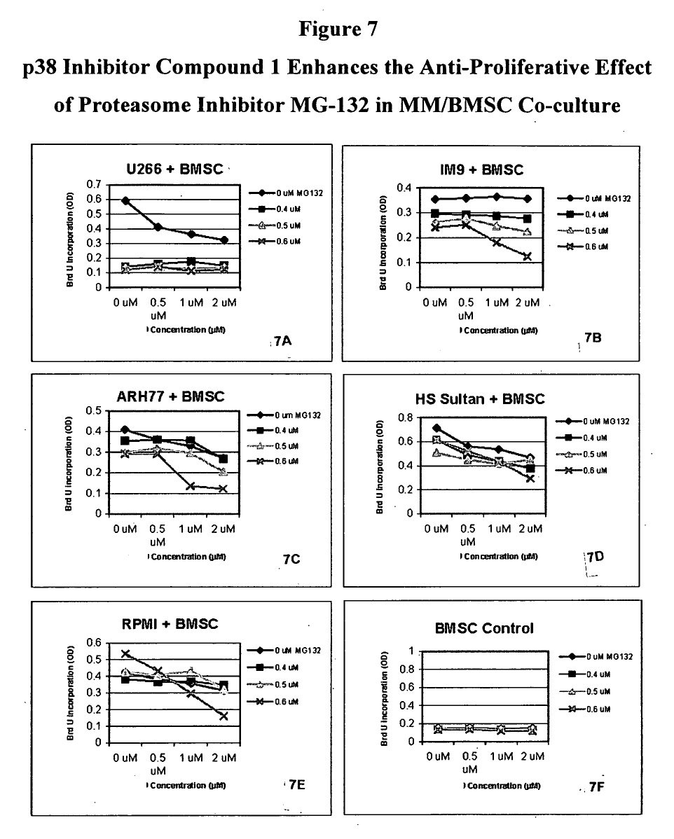

Treatment of BMSC with proteasome inhibitor MG-132 resulted in activation of p38. p38 activation is necessary for secretion of cytokines thought to cause resistance to cytotoxic chemotherapies such as proteasome inhibition. Thus, proteasome inhibitors such as VELCADE might be limiting their own ability to induce apoptosis of MM cells by inducing growth-supporting activity in the microenvironment. Inhibition of the p38 response by BMSC would counteract this support. In fact, exposure of MM/BMSC co-cultures to p38 MAPK inhibitor Compound 57 enhanced the ability of MG-132 to reduce MM cell proliferation (FIG. 7).

These interactions suggest potential benefits of co-treatment regimes in MM. Since dose limiting toxicities are observed with many treatments, the ability to achieve equivalent efficacy at lower doses, or to allow tolerance of a higher dose in order to further increase response, has clear potential for therapeutic benefit.

### Example 4

### p38 MAPK Inhibition Blocks Hsp27 Phosphorylation in MM Cells

Hsp27 has been implicated as an important factor in the development of drug resistance by MM cells. Therefore, Hsp27 is an attractive therapeutic target. Because Hsp27 is downstream of p38 MAP kinase in a signaling cascade (p38→MAPKAPK-2→Hsp27), an attempt is made to determine whether Hsp27 phosphorylation can be inhibited with the p38 MAPK inhibitor Compound 57 of Table 2.

For these studies, U266, IM9 and RPMI8226 cells are incubated with DMSO (−) or with 0.5 μM of a disclosed p38 MAPK inhibitor (+) for 1 hour and cell lysates are immunoblotted with antibodies to phospho-p38 and p38 MAP kinase by Western analysis. U266B1 and RPMI8226 are MM cell lines, and IM9 is an Epstein Barr Virus (EBV)-transformed B cell line with characteristics of MM cells. All can be obtained from American Type Culture Collection (ATCC; Rockville, Md.). All cell lines are maintained in RPMI-1640 (ATCC), supplemented with 10% fetal bovine serum (Hyclone; Logan, Utah), 100 units/ml of penicillin, 100 μg/ml streptomycin and 2 mM L-glutamine (Life Technologies, Inc.; Grand Island, N.Y.). BMSC (Cambrex) are maintained in Myelocult H5100 supplemented with 10⁻⁶ M hydrocortisone (Stem Cell Technologies; Vancouver, B.C.), 100 units/ml of penicillin and 100 μg/ml streptomycin. MM cells are usually seeded at a density of 3×10⁴ cells/well in 96-well culture plates. For MM co-cultures, BMSC are first seeded at 1.2×10⁴ cells/well in a 96-well plate in Myelocult/hydrocortisone medium for 24 hours prior to the addition of MM cells.

Total cell lysates are immunoprecipitated with anti-MAPKAPK-2 antibody and subjected to in vitro kinase assays using purified GST-Hsp27 as substrate. To examine Hsp27 phosphorylation in MM and transformed B cells, U266, IM9 and RPMI8226 cells are incubated either with DMSO or with 0.5 μM of a p38 MAPK inhibitor and cell lysates are immunoblotted with antibodies to phospho-Hsp27 (Ser 82) and Hsp27.

In additional experiments, the p38 MAPK inhibitors, such as Compound 57, are tested for their ability to inhibit Hsp27 phosphorylation. U266, IM9 and RPMI8226 cells are incubated with 0.5 μM of the p38 MAPK inhibitor for 1hour and Hsp27 proteins are immunoprecipitated with agarose-conjugated Hsp27 antibody, followed by immunoblotting with anti-phospho Hsp27 (Ser 78) antibody.

Specific antibodies to p38 MAP kinase are from Santa Cruz Biotechnology (Santa Cruz, Calif.). Antibodies to phospho-p38 MAP kinase (T180/Y182) and phospho-Hsp27 (S82) are from Cell Signaling (Beverly, Mass.). Antibodies to Hsp27 and phospho-Hsp27 (S78) are from Upstate Biotechnology (Lake Placid, N.Y.). Anti-MAPKAPK-2 is from StressGen (San Diego, Calif.) while anti-GAPDH is from Biogenesis Ltd. (Poole, UK).

Initially, the phosphorylation state of p38 in the MM cell lines U266 and RPMI8226 is examined as well as the EBV-transformed B cell line IM9, which has MM like characteristics. Interestingly, it is observed that these cells have high basal p38 phosphorylation levels and that stimulating these cells with TNFα does not lead to any appreciable increase in p38 phosphorylation or activation. Addition of the p38 MAPK inhibitor substantially suppresses p38 phosphorylation in all three cell lines. The p38 MAPK inhibitor blocks the catalytic activity of p38α but not the ability of p38 to act as a substrate for upstream MAPKKs (MKK3 and MKK6) nor do they indirectly inhibit MKK3 or MKK6 activity. Thus, the reduced p38 phosphorylation may be the result of a disrupted feedback loop involving p38 kinase activity and is manifested in blocked autophosphorylation. Consistent with inhibiting p38 kinase activity, Compound 57 suppresses the activity of downstream substrate MAPKAPK-2, as measured in an in vitro kinase assay. Finally, Compound 57 inhibits Hsp27 phosphorylation, as determined by direct examination of cell lysates or in immunoprecipitation assays. While Compound 57 does not affect total Hsp27 levels in these cells even after prolonged incubation with these inhibitors. These outcomes should demonstrate that the p38 MAPK inhibitors described herein effectively inhibit the components of the p38 MAP kinase pathway.

### Example 5

### p38 MAPK Inhibition Enhances PS-341-Induced MM Cytotoxicity

PS-341 (Kᵢ=0.62 nM for the chymotrypsin-like site of the proteasome) is a more potent and a more specific proteasome inhibitor than MG132 (Kᵢ=4 nM). MG132 has activity against calpains, cathepsins, and other lysosomal enzymes at concentrations 10-fold or more above those used in the disclosed experiments. PS-341 was recently approved by the FDA for treatment of MM.

For these experiments, RPM18226 and IM9 cells are treated with increasing concentrations of the p38 MAPK inhibitor Compound 57 in the presence of 7.5 nM, 8.0 nM or 8.5 nM of PS-341. Cell metabolism is determined by MTS assay. A time-course analysis of apoptosis after treatment of MM cells with PS-341 with or without the p38 MAPK inhibitor Compound 57. U266 cells are incubated with 10 nM PS-341 alone or in combination with 1.0 μM of the individual inhibitors. Cells are collected and labeled with Annexin V-PE and 7-AAD, followed by flow cytometry. A p38 MAPK inhibitor dose response analysis of apoptosis with PS-341 is also conducted. U266 cells are incubated with DMSO (control, blue), 5 nM PS-341 (yellow) or 5 nM PS-341 with increasing concentrations of inhibitor Compound 57 for 24 hours. Cells are labeled with Annexin V-PE and 7-AAD and analyzed by flow cytometry. PS-341 is purchased from Millenium (Cambridge, Mass.).

Similar to the results with MG132, the p38 MAPK inhibitors enhance PS341-induced cell toxicity in RPMI8226 and IM9 cells. Note however that unlike MG132, PS-341 has a very narrow dose response range as a sole agent. Nonetheless, the enhancement by the p38 MAPK inhibitors of PS-341-induced apoptosis is also observable in U266 cells. In a time-course experiment, 5 nM PS-341, a concentration that is much lower than the clinically observed circulating concentration of 40-100 nM in MM patients is used. As with MG132, a p38 MAPK inhibitor, even at 1 μM, is able to enhance the apoptotic effect of PS-341 on U266 cells. Increasing the amount of the p38 MAPK inhibitors in combination with a single dose of PS-341 also shows a consistent p38 MAPK inhibitor-mediated enhancement in U266 cytotoxicity.

These results can be corroborated in another MM cell line (MM.1S) and in MM patient cells, even in those patient cells that are resistant to PS-341. In addition, inhibition of p38 MAP kinase expression using siRNA technology is shown to mimic the effects of the p38 MAPK inhibitor in augmenting MM cytotoxicity by PS-341. To confirm the biologic significance of p38 MAPK inhibition in PS-341-treated MM cells, p38 MAPK siRNA is transfected into MM.1S cells. After 36 hours incubation, transfectants are cultured for 24 hours in the presence of PS-341 (2.5 nM). Importantly, 40% growth inhibition is noted in p38 MAPK siRNA MM.1S transfectants after treatment with 2.5 nM PS-341. Therefore, inhibition of p38 MAPK augments PS-341-triggered cytotoxicity in MM cells. p38 MAPK siRNA was purchased from Dharmacon Inc. (Lafayette, Colo.).

p38 MAPK inhibitors enhanced phosphorylation of c-Jun NH20 terminal kinase (JNK) and augments cleavage of caspase-8 and poly(ADP)-ribose polymerase (PARP). Moreover, the p38 MAPK inhibitors downregulate PS-341-induced increases in G2/M phase cells, associated with downregulation of p21Cip1 expression. Importantly, the p38 MAPK inhibitor treatment augments cytotoxicity of PS-431 even against PS-431 resistant cell lines and patient MM cells. These studies therefore provide the framework for clinical trials of p38 MAPK inhibitors to enhance sensitivity and overcome resistance of PS-431, thereby improving patient outcome in MM.

### Example 8

### p38 MAPK Inhibition Enhances PS-341-induced HSP27 Downregulation in MM Cells

Hsp27 is a stress protein with well-documented anti-apoptotic properties. Its activity is directly regulated by p38 through MAPKAPK-2. Because p38 MAPK inhibitors enhanced the effects of PS-341 in activating apoptosis, the idea that inhibiting Hsp27 activity, either by preventing its phosphorylation or changing the expression level following p38 inhibition, can explain the potentiation of PS-341 cytotoxicity.

To study this proposition, RPM18226 cells are incubated with 10 nM PS-341 in the presence or absence of 0.5 μM of p38 MAPK inhibitor Compound 57. Cells are collected at various times and lysates are examined by Western Blot analysis using specific antibodies against particular proteins, with GAPDH as loading control. Specific antibodies to Hsp70 and Hsp90 are from Santa Cruz Biotechnology (Santa Cruz, Calif.).

The Western blots are examined for the total Hsp27 and phospho-Hsp27 levels at three different exposure times. It is found that some heat shock proteins, including Hsp27 and Hsp70, are induced 8 hours after PS-341 treatment. The induction of these proteins is likely a part of the stress response in reaction to the increased presence of unfolded intracellular proteins in RPMI8226 cells treated with PS-341. This is consistent with observations made previously in MM.1S cells. However, while Hsp27 expression is initially induced 8 hours after PS-341 treatment, Hsp27 protein levels are reduced at 12 and 16 hours after treatment. This phenomenon appears to be unique to Hsp27 since a decrease in the levels of Hsp70 or Hsp90 in these same samples is not observed. Surprisingly, combining the p38 MAPK inhibitor Compound 57 with PS-341 results in an enhanced reduction of total Hsp27 at 12 and 16 hours in RPMI8226 as well as in IM9 cells. The decrease of phospho-Hsp27 levels in the PS-341-treated cells is commensurate with the decrease of total Hsp27 protein in these cells. In addition, there is complete inhibition of Hsp27 phosphorylation at all time points examined after addition of both drugs, as seen in PS341/p38 MAPK inhibitor treated cells.

### Example 9

### p38 MAPK Inhibition Facilitates PS-341-induced Caspase Activation and PARP Cleavage in MM Cells

The experiments discussed below suggest that the reduction of Hsp27 upon exposure to PS-341 and a p38 MAPK inhibitor correlates with the increased cytotoxicity observed in MM cells. RPMI8226 cells are treated with the p38 MAPK inhibitor Compound 57 of Table 2or PS-341, either alone or in combination, and examined these cells for apoptosis with Annexin V-PE by flow cytometry. RPMI8226 cells are incubated with DMSO, 0.5 μm of inhibitor Compound 57, 10 nM PS-341 or with the combination (0.5 μM p38 MAPK inhibitor/10 nM PS-341). Cells are collected at indicated times and assayed.

Detection of apoptotic cells was performed by staining with Annexin V-Phycoerythrin (PE) and 7-Amino Actinomycin D (7-AAD) (BD Pharmigen; San Diego, Calif.). Samples are analyzed by dual color flow cytometry using a FACScan laser flow cytometer and Cell Quest software (Becton Dickinson; San Jose, Calif.). 7-AAD is a nucleic acid dye that is used to exclude nonviable cells in flow cytometric assays. Cells that are Annexin V-PE positive and 7-AAD negative are considered early apoptotic. Antibodies to caspase-3, -6, -7, -8, -9, and PARP are from Cell Signaling (Beverly, Mass.).

Protein lysates from RPMI8226 cells obtained at these time points are collected in parallel and examined for Hsp27 levels, caspase activation and PARP cleavage by Western blot analysis. Cells exposed to either vehicle (DMSO) or one of the various p38 MAPK inhibitors do not undergo apoptosis, as indicated by Annexin V binding under these conditions. In those cells treated with PS-341, early apoptotic cells are apparent at 8 hours after drug treatment, while about half the cells at 12 hours, and most by 16 hours, are found to be apoptotic. Treatment with both a p38 MAPK inhibitor and PS-341 results in an increased proportion of apoptotic cells as expected with treatment with PS-341 alone, in agreement with the data obtained for U266 cells. At 16 hours, the proportion of Annexin positive cells treated with both agents is 20% greater than with PS-341 alone and 65% greater than with a p38 MAPK inhibitor alone.

Western blot analysis of cell lysates from these treatments show successively increasing activation or cleavage of procaspases-8, -9, -3, -6, and -7 with increasing duration of PS-341 treatment. Furthermore, a proportional increase is observed for PARP cleavage. The activation of these apoptotic enzymes is coordinately enhanced by treatment with a p38 MAPK inhibitor. Remarkably, the increased caspase and PARP activation observed for each exposure time correlates with the decreasing levels of Hsp27 and closely parallels the degree of apoptosis occurring in these cells. This result shows that the degradation of Hsp27 in RPMI8226 cells correlates with the increased caspase and PARP activation, and may cause the enhanced cytotoxicity of MM cells observed with the combination therapy of PS-341 and a p38 MAPK inhibitor.

### DISCUSSION

A serious impediment to treatment of multiple myeloma is the propensity for patients to develop resistance to current therapies. The proteasome inhibitor PS-341 has shown remarkable anti-tumor activity, even inducing complete responses, in some relapsed refractory MM patients. However, about two thirds of patients do not respond to this therapy and most who do eventually develop resistance to PS-341. The elucidation, therefore, of the mechanisms by which proteasome inhibitors like PS-341 promotes MM apoptosis and of the process by which MM cells develop resistance, could provide the basis for developing alternative strategies for enhancing treatment effectiveness and durability through combination with other therapies.

The combined exposure of multiple myeloma cells to proteasome inhibitors MG132 or PS-341 and to a p38 MAPK inhibitor results in an enhanced apoptosis of MM and transformed B cells. As demonstrated above, PS-341 leads to reduced levels of the anti-apoptotic protein Hsp27 in MM and that the p38 MAPK inhibitor causes reduction in level of the same protein, in addition to blocking its phosphorylation. Co-administration of a proteasome inhibitor and p38. inhibitor enhances the activation of pro-apoptotic proteins such as caspases and PARP and induces MM and transformed B cell cytotoxicity.

p38 MAP kinase has been demonstrated to play an anti-apototic role in a number of cell types. Inhibiting p38 specifically induced caspase-3 mediated apoptosis in transformed follicular lymphoma (FL)-derived cell lines. In lipopolysaccharide (LPS)-treated macrophage-like cell line J774.1, specific inhibitors of p38 were similarly found to induce apoptotic cell death. In multiple myeloma, inhibition of p38 MAP kinase has been previously shown to abrogate both constitutive and MM adhesion-induced IL-6 and VEGF secretion from BMSCs. However, treatment with a p38 MAPK inhibitor of MM and transformed B cells alone or in co-culture with BMSC does not lead to an appreciable decrease in MM viability under normal culture conditions. Whether MM cells require these p38-dependent factors for survival in response to stress, or for drug resistance, has not been tested. p38 MAPK inhibitors however, potentiate the direct cytotoxic effects of PS-341 on MM cells through the induction of apoptotic pathways regulated by Hsp27. p38 MAPK inhibitors inhibit the phosphorylation of Hsp27 by preventing the phosphorylation and activation of MAPKAPK-2 by p38, although inhibiting Hsp27 phosphorylation alone is not sufficient to induce MM cytotoxicity. However, in addition to preventing phosphorylation of Hsp27, p38 MAPK inhibitors also enhance the downregulation of Hsp27 by PS-341 and enhance PS-341-induced apoptosis. p38 MAPK inhibitors may diminish Hsp27 levels as a direct consequence of inhibiting Hsp27 phosphorylation in MM cells, as reported in other cells. In neutrophils, for example, TNFα stimulated the induction of Hsp27 gene expression, and that specific inhibitors of p38 significantly inhibited this induction of Hsp27. The mechanism by which PS-341 acts on Hsp27 level is not known, but could be indirect through altered level of transcription factors controlling Hsp27 or through altered levels or activity of other proteases.

Microarray analysis has shown that PS-341 downregulates the expression of genes involved in key pathways that promote MM growth and survival such as IL-6 and members of the IGF-1 receptor signaling pathways, and controls expression of several molecules involved in promoting both extrinsic and intrinsic apoptotic pathways. For instance, PS-341 downregulates the expression of anti-apoptotic proteins Bcl-2, A1, cIAP-2, FLIP and XIAP, redolent of the effects in MM of directly inhibiting NFκK activity. Downregulation of Bcl-2 and A1 is associated with cytochrome c release from the mitochondria and leads to early cleavage of caspase-9. PS-341 also upregulates pro-apoptotic genes such as Bax, caspase-8, -9, -1, -7, -4 and -5, death-inducing receptors DR5 and Fas, as well as the death ligand FasL, thereby inducing the extrinsic apoptotic pathway regulated by caspase-8. PS-341 has also been shown to acutely upregulate several heat shock proteins including Hsp27, presumably as a normal stress response prompted by proteasome inhibition, to facilitate the removal of excess proteins and to stabilize the cytoskeleton. However, the induction of Hsp27 is seen only at early exposure times when MM cells have yet to undergo apoptosis. In MM and transformed B cells undergoing cell death, a direct correlation of enhanced apoptosis with decreased Hsp27 levels while other heat shock proteins such as Hsp70 and 90 remain unaltered is shown here.

The role of Hsp27 in providing cytoprotection is underscored by its role in preventing key steps along the pro-apoptotic pathway. For instance, Hsp27 inhibits the intrinsic pathway of apoptosis by blocking the release of cytochrome c from the mitochondria and preventing the formation of critical components of the apoptosome, thus negatively regulating the activation of procaspase-9. As a molecular chaperone, Hsp27 inhibits caspase-3 activation by interacting with procaspase-3, and preventing its activation by caspase-9. Hsp27 has also been associated with the inhibition of the extrinsic apoptosis initiated by death ligands such as FasL through its interaction with procaspase-3, thus preventing caspase-8-mediated response. Furthermore, Hsp27 is involved in caspase-independent apoptosis through its interaction with Daxx, which prevents the recruitment of Ask1, a MAPKKK that activates the JNK pathway. Hsp27 has also been shown to associate and activate protein kinase B (Akt), which adds increased resistance to apoptosis observed in cells expressing high levels of Hsp27.

Over-expression of Hsp27 has been demonstrated to promote resistance to PS-341 in some lymphoma cells. In multiple myeloma, MM cells expressing high levels of Hsp27 have also been found to be refractory to dexamethasone. Treating patient cells with biochemical inhibitors of Hsp27 such as Quercitin or reducing Hsp27 expression through anti-sense technology led to a significant amelioration of dexamethasone sensitivity. Since the combined exposure of MM and transformed B cells to both PS-341 and p38 MAPK inhibitors reduces Hsp27, this treatment is predicted to reverse resistance of MM cells to dexamethasone. Similar response might also be obtained in other Hsp27-overexpressing cancers. Thus, in addition to the complementary effects of p38 MAPK inhibitors and PS-341 in blocking microenvironment production of factors supporting MM cell growth, proliferation and survival, the combined therapy with p38 MAPK inhibitors enhance the cytotoxic effects of PS-341 on MM cells, providing the basis for an improved therapy for multiple myeloma.

### Example 10

### Inhibitors of p38a MAPK and Proteasome Inhibitors for Treating a Subject with Multiple Myeloma

A patient is diagnosed with multiple myeloma. The patient presents with MM cells with rapid growth rates, which displace osteoblasts, and disrupting the balance of bone creation and destruction. A number of MM-related cytokines, such as IL-6, VEGF, IL-11, and PGE-2 are detectable. A therapeutic amount of p38 MAPK inhibitor Compound 57 in the hydrochloric salt form in combination with VELCADE is administered. MM cell growth is inhibited and MM-related cytokine product is reduced.

### Example 11

### Treating a Subject with VELCADE-Resistant Multiple Myeloma

A patient diagnosed with multiple myeloma receives VELCADE and responds well until VELCADE-resistant MM cells develop. Drug resistant MM cells with rapid growth rates begin to populate the bone marrow of the subject. Elevated levels of MM-related cytokines, such as IL-6, VEGF, IL-11, and PGE-2 are again detectable. A therapeutic amount of p38 MAPK inhibitor Compound 57 in the hydrochloric salt form in combination with VELCADE is administered. The combination restores the sensitivity of the MM cells to VELCADE. MM cell growth is again inhibited and MM-related cytokine product is reduced.

What is claimed is:

1. A method to treat multiple myeloma in a subject, comprising:
co-administering to a subject in need of such treatment a therapeutically effective amount of a p38 inhibitor and a therapeutically effective amount of a proteasome inhibitor, whereby a symptom associated with multiple myeloma is ameliorated.

2. The method of claim 1, wherein the p38 inhibitor is of the formula: 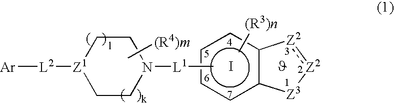
and the pharmaceutically acceptable salts thereof, or a pharmaceutical composition thereof, wherein 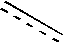
represents a single or double bond;
one Z² is CA or CR⁶A and the other is CR¹ or CR¹₂, wherein each R¹ is independently hydrogen is alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof;
R⁶ is H, alkyl, alkenyl, alkynyl, aryl, arylalkyl, acyl, aroyl, heteroaryl, heteroalkyl, heteroalkenyl, heteroalkynyl, heteroalkylaryl, SOR, SO₂R, RCO, COOR, alkyl-COR, SO₃R, CONR₂, SO₂NR₂, CN, CF₃, or R₃Si wherein each R is independently H, alkyl, alkenyl or aryl or the heteroforms thereof;
A is —Wᵢ—COXⱼY wherein Y is COR² wherein R² is hydrogen, straight or branched chain alkyl, alkenyl, alkynyl, aryl, arylalkyl, heteroalkyl, heteroaryl, or heteroarylalkyl, each optionally substituted with halo, alkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroforms thereof; or
wherein R² is OR, NR₂, NRCONR₂, OCONR₂, NRSO₂NR₂, heteroarylalkyl, COOR, NRNR₂, heteroaryl, heteroaryloxy, heteroaryl-NR, or NROR wherein each R is independently H, alkyl, alkenyl or aryl or the heteroforms thereof, and wherein two R attached to the same N atom may form a 3-8 member ring and wherein said ring may further be substituted by alkyl, alkenyl, alkynyl, aryl, arylalkyl, heteroaryl, heteroalkyl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroforms thereof wherein two R attached to the same atom may form a 3-8 member ring, optionally substituted as above defined, and
each of W and X is substituted or unsubstituted alkylene or alkenylene, each of 2-6 Å, or
Y is tetrazole; 1,2,3-triazole; 1,2,4-triazole; or imidazole;
each of i and j is independently 0 or 1;
Z³ is NR⁷ or O;
R⁷ is H or is optionally substituted alkyl, alkenyl, alkynyl, aryl, arylalkyl, acyl, aroyl, heteroaryl, heteroalkyl, heteroalkenyl, heteroalkynyl, or heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof;
each R³ is independently halo, alkyl, heteroalkyl, OCOR, OR, NRCOR, SR, or NR₂, wherein R is H, alkyl or aryl or the heteroforms thereof;
n is 0-3;
L¹ is CO, SO₂ or alkylene (1-4C);
L² is alkylene (1-4C) or alkenylene (2-4C) optionally substituted with one or two moieties selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, and R₃Si, wherein each R is independently H, alkyl, alkenyl or aryl or the heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety;
each R⁴ is independently selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof;
m is 0-4;
Z¹ is CR⁵ or N wherein R⁵ is hydrogen or OR, NR₂, SR or halo, wherein each R is independently H, alkyl, alkenyl or aryl or the heteroforms thereof;
each of 1 and k is an integer from 0-2 wherein the sum of 1 and k is 0-3;
Ar is an aryl group substituted with 0-5 noninterfering substituents selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and wherein two of said optional substituents on adjacent positions can be joined to form a fused, optionally substituted aromatic or nonaromatic, saturated or unsaturated ring which contains 3-8 members.

3. The method of claim 1, wherein the proteasome inhibitor is selected from the group consisting of epoxomicin ((2R)-2-[Acetyl-(N-Methyl-L-Isoleucyl)-L-Isoleucyl-L-Threonyl-L-Leucyl]-2-Methyloxirane); lactacystin(N-Acetyl-L-Cysteine, S-[2R,3S,4R]-3-Hydroxy-2-[(1S)-1-Hydroxy-2-Methylpropyl]-4-Methyl-5-Oxo-2-Pyrolidinecarbonyl]); Z-Ile-Glu(OtBu)-Ala-Leu-H(Carbobenzoxy-L-Isoleucyl-Gamma-t-Butyl-L-Glutamyl-L-Alanyl-L-Leucinal; Z-Leu-Leu-Leu-H [MG 132](Carbobenzoxy-L-Leucyl-L-Leucyl-L-Leucinal); and Z-Leu-Leu-Nva-H [MG 115] (Carbobenzoxy-L-Leucyl-L-Leucyl-L-Norvalinal).

4. The method of claim 1, wherein the proteasome inhibitor is bortezombid (VELCADE), thalidomide or REVIMID.

5. The method of claim 1, wherein the symptom comprises a rate of MM cell growth which is reduced as compared to the MM cell growth rate of untreated MM cells.

6. The method of claim 1, wherein the symptom comprises production of a MM-related cytokine, which is reduced.

7. The method of claim 6, wherein the MM-related cytokine is selected from the group consisting of IL-6, VEGF, IL-11, and PGE-2.

8. The method of claim 1, wherein the p38 MAPK inhibitor and the proteasome inhibitor are administered simultaneously.

9. A method of inhibiting cytokine secretion from multiple myeloma cells, comprising:
providing a p38 MAP kinase inhibitor and a proteasome inhibitor a subject suffering from MM, wherein the secretion rate of a MM-related cytokine is reduced as compared to the secretion rate of a MM-related cytokine of untreated MM cells.

10. The method of claim 9, wherein the MM-related cytokine is selected from the group consisting of IL-6, VEGF, IL-11, and PGE-2.

11. The method of claim 9, wherein the p38 inhibitor is of the formula: 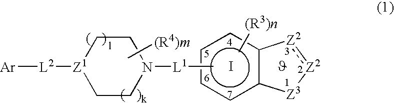
and the pharmaceutically acceptable salts thereof, or a pharmaceutical composition thereof, wherein 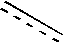
represents a single or double bond;
one Z² is CA or CR⁸A and the other is CR¹, CR¹₂, NR⁶ or N wherein each R¹, R⁶ and R⁸ is independently hydrogen or noninterfering substituent;
A is —Wᵢ—COXⱼY wherein Y is COR² or an isostere thereof and R² is hydrogen or a noninterfering substituent, each of W and X is a spacer of 2-6 Å, and each of i and j is independently 0 or 1;
Z³ is NR⁷ or O;
each R³ is independently a noninterfering substituent;
n is 0-3;
each of L¹ and L² is a linker;
each R⁴ is independently a noninterfering substituent;
m is 0-4;
Z¹ is CR⁵ or N wherein R⁵ is hydrogen or a noninterfering substituent;
each of 1 and k is an integer from 0-2 wherein the sum of 1 and k is 0-3;
Ar is an aryl group substituted with 0-5 noninterfering substituents, wherein two noninterfering substituents can form a fused ring.

12. The method of claim 9, wherein the proteasome inhibitor is selected from the group consisting of epoxomicin ((2R)-2-[Acetyl-(N-Methyl-L-Isoleucyl)-L-Isoleucyl-L-Threonyl-L-Leucyl]-2-Methyloxirane); lactacystin(N-Acetyl-L-Cysteine, S-[2R,3S,4R]-3-Hydroxy-2-[(1S)-1-Hydroxy-2-Methylpropyl]-4-Methyl-5-Oxo-2-Pyrolidinecarbonyl]); Z-Ile-Glu(OtBu)-Ala-Leu-H(Carbobenzoxy-L-Isoleucyl-Gamma-t-Butyl-L-Glutamyl-L-Alanyl-L-Leucinal; Z-Leu-Leu-Leu-H [MG 132](Carbobenzoxy-L-Leucyl-L-Leucyl-L-Leucinal); and Z-Leu-Leu-Nva-H [MG 115] (Carbobenzoxy-L-Leucyl-L-Leucyl-L-Norvalinal).

13. The method of claim 9, wherein the proteasome inhibitor is bortezombid (VELCADE), thalidomide or REVIMID.

14. The method of claim 9, wherein the p38 MAPK inhibitor and the proteasome inhibitor are administered simultaneously.

15. A method of overcoming drug resistance in MM tumor cells comprising:
identifying a drug resistant MM tumor cell; and
providing a p38 MAPK inhibitor and a chemotherapeutic agent effective in inhibiting MM cell growth, whereby a synergistic interaction of the p38 MAPK inhibitor and the chemotherapeutic agent overcome the drug resistance of the MM tumor cells.

16. The method of claim 15, wherein the p38 inhibitor is of the formula: 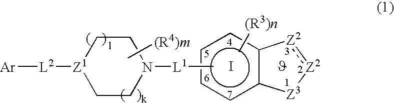
and the pharmaceutically acceptable salts thereof, or a pharmaceutical composition thereof, wherein 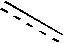
represents a single or double bond;
one Z² is CA or CR⁸A and the other is CR¹, CR¹₂, NR⁶ or N wherein each R¹, R⁶ and R⁸ is independently hydrogen or noninterfering substituent;
A is —Wᵢ—COXⱼY wherein Y is COR² or an isostere thereof and R² is hydrogen or a noninterfering substituent, each of W and X is a spacer of 2-6 Å, and each of i and j is independently 0 or 1;
Z³ is NR⁷or O;
each R³ is independently a noninterfering substituent;
n is 0-3;
each of L¹ and L² is a linker;
each R⁴ is independently a noninterfering substituent;
m is 0-4;
Z¹ is CR⁵ or N wherein R⁵ is hydrogen or a noninterfering substituent;
each of 1 and k is an integer from 0-2 wherein the sum of I and k is 0-3;
Ar is an aryl group substituted with 0-5 noninterfering substituents, wherein two noninterfering substituents can form a fused ring.

17. The method of claim 15, wherein the proteasome inhibitor is selected from the group consisting of epoxomicin((2R)-2-[Acetyl-(N-Methyl-L-Isoleucyl)-L-Isoleucyl-L-Threonyl-L-Leucyl]-2-Methyloxirane); lactacystin(N-Acetyl-L-Cysteine, S-[2R,3S,4R]-3-Hydroxy-2-[(1S)-1-Hydroxy-2-Methylpropyl]-4-Methyl-5-Oxo-2-Pyrolidinecarbonyl]); Z-Ile-Glu(OtBu)-Ala-Leu-H(Carbobenzoxy-L-Isoleucyl-Gamma-t-Butyl-L-Glutamyl-L-Alanyl-L-Leucinal; Z-Leu-Leu-Leu-H [MG 132](Carbobenzoxy-L-Leucyl-L-Leucyl-L-Leucinal); and Z-Leu-Leu-Nva-H [MG 115] (Carbobenzoxy-L-Leucyl-L-Leucyl-L-Norvalinal).

18. The method of claim 15, wherein the proteasome inhibitor is bortezombid (VELCADE), thalidomide or REVIMID.

19. The method of claim 15, wherein the p38 MAPK inhibitor and the proteasome inhibitor are administered simultaneously.

20. The method of claim 15, wherein the p38 MAPK inhibitor and the chemotherapeutic agent are provided to a subject containing one or more drug resistant MM tumor cells.
